(19) United States
(12) Patent Application Publication
(10) Pub. No.: US 2024/0285656 A1
(43) Pub. Date: Aug. 29, 2024

(54) CANNABIDIOL (CBD) AND TERPENE FORMULATION THAT INCREASES RESTORATIVE SLEEP IN HUMANS

(71) Applicant: Defined Research, Inc., San Francisco, CA (US)

(72) Inventors: Paul Muchowski, San Francisco, CA (US); Eric Chang, San Francisco, CA (US); Michael Wang, San Francisco, CA (US)

(21) Appl. No.: 18/659,499

(22) Filed: May 9, 2024

Related U.S. Application Data
(63) Continuation of application No. PCT/US2023/011720, filed on Jan. 27, 2023.
(60) Provisional application No. 63/303,656, filed on Jan. 27, 2022.

(51) Int. Cl. A61K 31/00; A61K 9/48; A61K 31/01; A61K 31/015; A61K 31/045; A61P 25/20

(52) U.S. Cl. CPC A61K 31/658; A61K 9/4816; A61K 31/01; A61K 31/015; A61K 31/045; A61P 25/20

(57) ABSTRACT

A Cannabidiol (CBD) and terpene formulation that increases restorative sleep and decreases anxiety and depression in humans is disclosed herein.

### CROSS-REFERENCE TO RELATED APPLICATIONS

The present application claims priority from and is a continuation from PCT application serial no. PCT/US2023/011720, filed Jan. 27, 2023, which claims priority from U.S. provisional application Ser. No. 63/303,656, filed Jan. 27, 2022, each herein incorporated by reference in their entireties.

### BACKGROUND

The invention generally relates to a novel formulation of CBD and terpenes and its use for the treatment of disease, and, more specifically, to a formula and their use for the treatment of sleep related disorders.

Insomnia is a disorder in which people have inadequate or poor-quality sleep due to a number of factors, such as difficulty falling asleep, waking up frequently during the night with difficulty returning to sleep, waking up too early in the morning, or having unrefreshing sleep.

A survey of 1,267 adults by Consumer Reports indicates that nearly 80% of Americans have trouble sleeping at least once a week. Insomnia is the most common sleep disorder and is an established risk factor for anxiety, depression and other diseases. Insomnia is defined clinically as the perception or complaint of inadequate or poor-quality sleep due to a number of factors, such as difficulty falling asleep, waking up frequently during the night with difficulty returning to sleep, waking up too early in the morning, or having unrefreshing sleep. Insomnia causes significant distress and/or impairment in daytime functioning in people who suffer from it. The prevalence rates of clinically diagnosed insomnia have increased in recent years to ˜19% of the adult U.S. population, representing ˜46 million. Some studies suggest that the rate of undiagnosed insomnia in the US may be as high as 25% (˜80 million)¹.

CBD is the active ingredient of Epidiolex®, a drug approved by the FDA in 2018 for the treatment of seizures associated with two rare and severe forms of epilepsy, Lennox-Gastaut syndrome and Dravet syndrome. In 2018, the U.S congress also passed the Hemp Farming Act of 2018 (the Farm Bill), which removed remove hemp (defined as cannabis with less than 0.3% THC) from Schedule I controlled substances, and made it an ordinary agricultural commodity. This Bill immediately led to the widespread purification, sale and interstate commerce of CBD, a molecule obtained from hemp ($4.7 billion in U.S. sales in 2021). The result has been that storefronts and online retailers have flooded the market with CBD products, many with unsubstantiated therapeutic claims (for example, for treating insomnia). Sadly, U.S. consumers are currently using CBD to treat a variety of ailments in the absence of rigorous clinical studies. Indeed, ˜14% of the U.S. adult population (˜29 million adults) admit to using CBD (Gallup Survey, U.S. News & World Report). Nonetheless, rigorous clinical research is desperately lacking to support the use of CBD for the numerous ailments that Americans are ostensibly using it to treat. The FDA's biggest concern is the marketing of CBD products that make unsubstantiated therapeutic claims to prevent, diagnose, mitigate, treat, or cure serious diseases, but have not obtained new drug approvals.

In a recent survey by Consumer Reports, ˜40% of adults who reported trying CBD said they used it to help them sleep, and a majority of those people said they believed that it worked. Nonetheless, to this date a rigorous, properly-controlled, and well-powered study to determine if CBD influences sleep physiology has simply never been reported in the scientific literature.

The present invention attempts to solve these problems as well as others.

### SUMMARY OF THE INVENTION

Provided herein are methods and compositions for increasing restorative Sleep in Humans.

The CBD formulation is composed of CBD and a plurality of terpenes including linalool, myrcene, phytol, limonene, α-terpinene, α-terpineol, α-pinene, and β-caryophyllene to treat insomnia and other sleep disorders including pain, anxiety and depression.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is a circle graph showing the GABA_{A} receptor and binding sites.

FIGS. 2A-2D are graphs showing the CBD formulation increases (FIG. 2A) REM and (FIG. 2B) Restorative Sleep in study participant DR11, as well as the percentage of time DR11 spends in (FIG. 2C) REM and (FIG. 2D) restorative sleep [**p<0.01 (t test). n=7 days per treatment group. Values are means±SEM].

FIGS. 3A-3B are graphs showing the CBD formulation increases the percentage of time spent in (FIG. 3A) REM and (FIG. 3B) Restorative Sleep in study participant DR11, but CBD alone is less effective [*p<0.05 (One-way ANOVA). n=7 days per treatment group. Values are means±SEM].

FIG. 4A is a graph showing the CBD formulation shows a trend to increase the percentage of REM sleep [p=0.06; (t test). n=23 participants per treatment group. Values are means±SEM]; and FIG. 4B is a graph showing the CBD formulation trend to increase the percentage of restorative sleep in pooled data from Le Rêve 1.0 [p=0.32 (t test). n=23 participants per treatment group. Values are means±SEM].

FIGS. 5A-5B are graphs showing the CBD formulation increases the percentage of (FIG. 5A) REM and (FIG. 5B) Restorative Sleep in pooled responders [**p=0.001; ****p<0.0001 (t test). n=11 responders per treatment group. Values are means±SEM].

FIGS. 6A-6B are graphs showing the CBD formulation increases the percentage of (6A) REM in participants with moderate insomnia and (6B) Restorative Sleep in pooled responders with insomnia [**p<0.01; (t test). n=7-9 responders per treatment group. Values are means±SEM].

FIG. 7 is a graph showing the Study Participants Perceived That the CBD formulation Helped Them Sleep Better [p=0.13; (t test). n=23 responders per treatment group. Values are means±SEM]. At the end of each treatment period, study participants used a PGI questionnaire to determine if the treatment influenced their sleep relative to a baseline period before the treatments were initiated. Scoring Matrix: 1=Treatment Helped; 2-No Effect of Treatment; 3-Treatment Worsened. With this data, a lower score indicates a potential benefit to help sleep. The results showed that the CBD formulation appeared to improve the perception of better sleep relative to the placebo control.

FIGS. 8A-8C are graphs showing the CBD formulation Decreasing Anxiety. At the end of each treatment period, study participants completed a clinically-validated survey entitled the Hamilton Anxiety Rating Scale²⁹. Each item in this rating scale is scored on a scale of 0 (not present) to 4 (severe), with a total score range of 0-53, where <14 indicates mild severity, 15-21 mild to moderate severity, and 22-27 moderate to severe. Based on this survey, the CBD formulation decreases anxiety in participants DR13 (FIG. 8A), DR16 (FIG. 8B), and DR21 (FIG. 8C) relative to the placebo control or CBD alone.

FIG. 9A is a graph showing the CBD formulation decreases Anxiety in “Responders”. At the end of each treatment period, study participants completed a survey entitled the Hamilton Anxiety Rating Scale²⁹. Each item in this rating scale is scored on a scale of 0 (not present) to 4 (severe), with a total score range of 0-53, where <14 indicates mild severity, 15-21 mild to moderate severity, and 22-27 moderate to severe. Based on this survey, the CBD formulation appeared to decrease anxiety relative to the placebo control or CBD alone [*p<0.05 (One-way ANOVA). n=10 “responders” per treatment group. Values are means±SEM]. FIG. 9B is a graph showing the average anxiety score in responders is higher than non-responders after taking the placebo control [*p<0.05 (t test). n=10-11 participants per treatment group. Values are means ±SEM].

FIGS. 10A-10B are graphs showing the CBD formulation increases the percentage of (10A) REM and (10B) restorative sleep in an independent cohort of participants with moderate to severe insomnia [*p<0.05; (t test). n=13 participants per treatment group. Values are means ±SEM].

FIGS. 11A-11B are graphs showing the CBD formulation increases the percentage of (11A) REM and (11B) restorative sleep in a combined data set from clinical studies 1 and 2 [*p<0.05; (t test). n=36 participants per treatment group. Values are means±SEM]. FIG. 11C is a graph showing the CBD formation shows A Trend To Increase The Percentage Of Deep Sleep In Combined Data From Le Rêve 1.0 and 2.0 [p=0.17 (t test). n=36 participants per treatment group. Values are means±SEM].

FIG. 12 is a schematic flow chart of the clinical study for the examples.

FIGS. 13A-13F are graphs showing the CBD-terpenes formulation increases the percentage of time DR24 (FIG. 13A), DR45 (FIG. 13B), DR51 (FIG. 13C), DR111 (FIG. 13D), DR112 (FIG. 13E), and DR128 (FIG. 13F) spends in SWS+REM sleep (restorative sleep) [*p<0.05 (t test); **p<0.01 (t test); ***p<0.001; ****p<0.0001 (t test). n=15-28 days per treatment group. Values are means±SEM].

FIGS. 14A-14F are graphs showing the CBD-terpenes formulation increases (FIG. 14A) REM, (FIG. 14B) SWS, (FIG. 14C) SWS+REM (restorative sleep) in study participant DR51, as well as the percentage of time DR51 spends in (FIG. 14D) REM and (FIG. 14E) SWS, and (FIG. 14F) SWS+REM (restorative sleep) [**p<0.01; ***p<0.001 (t test). n=19-25 days per treatment group. Values are means±SEM].

FIGS. 15A-15F are graphs showing the CBD-terpenes formulation increases (FIG. 15A) REM, (FIG. 15B) SWS, (FIG. 15C) SWS+REM (restorative sleep) in study participant DR128, as well as the percentage of time DR128 spends in (FIG. 15D) REM and (FIG. 15E) SWS, and (FIG. 15F) SWS+REM (restorative sleep)*p<0.05 (t test); ***p<0.001; ****p<0.0001 (t test). n=15-28 days per treatment group. Values are means±SEM].

FIGS. 16A-16F are graphs showing the CBD-terpenes formulation decreases light sleep compared to placebo in study participant DR24 (FIG. 16A), in study participant DR45 (FIG. 16B), in study participant DR51 (FIG. 16C), in study participant DR111 (FIG. 16D), in study participant DR112 (FIG. 16E), and study participant DR128 (FIG. 16F) *p<0.05 (t test); ***p<0.001; ****p<0.0001 (t test). n=15-28 days per treatment group. Values are means±SEM].

FIGS. 17A-17F are graphs showing the CBD-terpenes formulation increases total sleep time (TST) compared to placebo in study participant DR24 (FIG. 17A), in study participant DR45 (FIG. 17B), in study participant DR51 (FIG. 17C), in study participant DR111 (FIG. 17D), in study participant DR112 (FIG. 17E), and study participant DR128 (FIG. 17F) [(t test); n=15-28 days per treatment group. Values are means±SEM], “ns” not significant.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

“Sleep disorder” is any condition where a subject has difficulty falling asleep and difficulty staying asleep such as insomnia, and includes conditions related to difficulty falling asleep due to anxiety, stress, and depression; difficulty sleeping resulting in poor concentration and focus; difficulty falling asleep due to age-related memory loss, dementia, or other cognitive disorder; difficulty falling asleep due to substance abuse, mental disorders, breathing disorders, or by other sleep disorders such as periodic limb movement; difficulty falling asleep or staying asleep due to other sleep related problems such as poor sleep hygiene, poor sleep hygiene includes consumption of beverages with alcohol or caffeine, eating large meals, or engaging in physically or mentally stimulating activity shortly before bed time; poor sleep hygiene includes a highly variable bed time, or inadequate temperature, poor ventilation, noise or light within the sleep environment; including narcolepsy, and Rapid Eye Movement (REM) sleep behavior disorder; disorders associated with neurodegenerative diseases such as Alzheimer's disease, Parkinson's disease, and multiple sclerosis; disrupted REM sleep disorder associated with drug withdrawal, especially alcohol or sedative-hypnotic withdrawal; and disrupted circadian rhythm associated with sleep apnea, shift work and jet lag.

The term “anxiety” as used herein refers to mental disorders in which anxiety and avoidance behavior predominate. Examples of such disorders include phobias (including agoraphobia), panic disorder, obsessive-compulsive disorder, posttraumatic stress disorder, acute stress disorder, generalized anxiety disorder, and substance-induced anxiety disorder.

“Depression” includes a morbid or clinical depression, which is usually diagnosed when sadness or elation is overly intense and continues beyond the expected impact of an event. Symptoms often recur on an episodic basis or pursue a low-grade intermittent chronicity, which impairs the functioning of the sufferer. Bipolar mood disorder, which commonly begins with depression and is characterised by periods of elation during the course of the illness. Unipolar mood disorder, which is characterised as syndromal depression of episodes that last for typically 6 to 9 months.

“REM sleep” is characterized by rapid eye movement, muscular atonia, dream content, fluctuations in autonomic function (irregular respiration, pulse, temperature, and blood pressure), a brain metabolic rate similar to waking, and desynchronized neuronal activity. There is neither noradrenergic activity nor serotonergic activity during REM. REM sleep does not serve the same purpose (neuronal activity is similar to that of waking activity), but it may serve yet another role: the restoring full sensitivity of monoamine receptors (especially those for norepinephrine, serotonin and histamine). This is accomplished by the cessation of monoamine neurotransmitter release during REM sleep (causing sleep paralysis and reducing environmental awareness). Other theories related to this hypothesis argue that REM sleep is important in memory (especially memory consolidation) and intellectual function. Studies show that REM sleep increases during intense learning experiences and that REM sleep deprivation leads to reduced creative problem solving ability. Experiments have shown that perceptual skills, such as those that are learned through repeated practice, improve overnight and are disrupted if there is selective interruption of REM sleep. Other experimental data suggest that cerebral activation that occurs during REM sleep plays a key role in brain development.

REM sleep is additionally linked to proper functioning of active growth and development of the nervous system. The fact that REM sleep is resistant to age-related changes is believed to suggest a role in maintaining nervous system function. Moreover, while the purpose of dreaming (a key distinguishing feature of REM) is even less well understood than REM, many the theories about it reinforce the cognitive-health importance of REM sleep. These theories include proposing that dreaming (and thus REM) is intertwined with long-term memory consolidation of semantic memories, learning, and resolution of distressing experiences.

“Restorative Sleep” is the general implication is that sleep plays an important role in revitalization. Mechanisms underlying the restoration process include neutralization of neurotoxins that accumulate during waking hours, responses to increased sleep-inducing substances that are produced during waking processes, neurochemical synthesis, and brain chemical redistribution. During slow wave sleep (SWS), increased tissue synthesis, cell division, and growth hormone release is observed. Athletes have higher proportions of SWS than others. Oxygen consumption declines during SWS suggesting reduced catabolism. SWS increases after starvation in an apparent compensatory effect. Hyperthyroidism increases SWS whereas hypothyroidism reduces SWS, and SWS is high during peak physical developmental years in children and declines during advancing age. Last, research indicates that SWS has an intensity component. This intensity dimension of SWS apparently allows mammals to compensate for lost sleep without having to significantly increase sleep time. While NREM sleep, and in particular SWS appear to play a central role in physical restoration. The lower metabolic rates and lower body temperatures of NREM apparently provide an environment conducive to neuronal repair.

The term “synergistic” as used herein is refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-anxiety; ingredient B is pro-anxiety; when A and B are combined, said combination produces an anti-anxiety effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

The term “CBD formulation” as used herein refers to a composition which is pharmaceutically acceptable and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. CBD formulation includes configurational isomers (such as cis and trans isomers) and all optical isomers (such as enantiomers) Isomers and diastereomers), racemic, diastereoisomers and other mixtures of these isomers, as well as solvates, hydrates, isomorphs, polymorphs, tautomers, Ester, salt forms and prodrugs. The term “prodrug” refers to a compound that is a drug precursor, which releases the drug in vivo through some chemical or physiological processes after administration (for example, the prodrug is transformed into the desired drug form when it reaches physiological pH or through the action of enzymes). Exemplary prodrugs release the corresponding free acid upon cleavage, and the hydrolyzable ester-forming residues of the compounds of the present invention.

In the present disclosure, an “effective amount” or an “effective dose” of CBD, terpenes, combination of CBD with terpenes, or composition refers to an amount of CBD or terpenes that, once administered to a subject, will reach the subject's bloodstream and/or bodily tissues.

### CBD

It should be appreciated that in the context of the present invention the terms “cannabidiol compound”, “cannabidiol” or “CBD” (which may be used interchangeably unless the context clearly dictates otherwise) refer to any natural, semi-synthetic or synthetic cannabinoid compound.

CBD, unless a particular other stereoisomer or stereoisomers are specified, includes the compound “Δ²-cannabidiol.” These compounds are: (1) Δ⁵-cannabidiol (2-(6-isopropenyl-3-methyl-5-cyclohexen-1-yl)-5-pentyl-1,3-benzenediol); (2) Δ⁴-cannabidiol (2-(6-isopropenyl-3-methyl-4-cyclohexen-1-yl)-5-pentyl-1,3-benzenediol); (3) Δ³-cannabidiol (2-(6-isopropenyl-3-methyl-3-cyclohexen-1-yl)-5-pentyl-1,3-benzenediol); (4) Δ^{3,7}-cannabidiol (2-(6-isopropenyl-3-methylenecyclohex-1-yl)-5-pentyl-1,3-benzenediol), (5) Δ²-cannabidiol (2-(6-isopropenyl-3-methyl-2-cyclohexen-1-yl)-5-pentyl-1,3-benzenediol); (6) Δ¹-cannabidiol (2-(6-isopropenyl-3-methyl-1-cyclohexen-1-yl)-5-pentyl-1,3-benzenediol); and (7) Δ⁶-cannabidiol (2-(6-isopropenyl-3-methyl-6-cyclohexen-1-yl)-5-pentyl-1,3-benzenediol).

CBD may include cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannahivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids. Each possibility represents a separate embodiment of the invention.

According to one embodiment, the CBD formulation comprises the following general Formula (I):

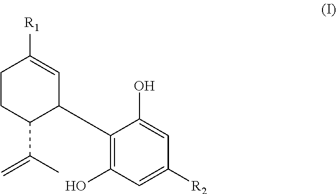

Wherein R1 is an alkyl; and

R2 is selected from a straight or branched alkyl having 5 to 12 carbon atoms; an —OR3 group, wherein R3 is a straight or branched alkyl having 5 to 9 carbon atoms or a straight or branched alkyl substituted at the terminal carbon atom by a phenyl group; or a —(CH₂)n-O-alkyl group, wherein n is an integer from 1 to 7 and the alkyl group has 1 to 5 carbons.

In one embodiment, R1 is CH₃ and R₂ is a straight alkyl having 5 carbon atoms (i.e. —C₅H₁₁).

In another embodiment, the CBD compound is cannabidiol. CBD, the molecular formula of C₂₁H₃₀O₂, as depicted in the following formula (II):

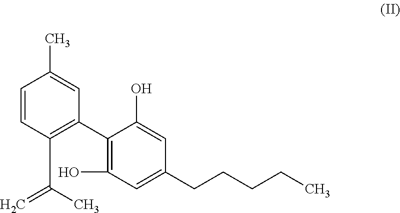 [SMILES: C=C(C)c1ccc(C)cc1-c1c(O)cc(CCCCC)cc1O]

The cannabidiol of formula (II) may be a natural cannabidiol obtainable by extraction from a plant member of the genus Cannabis or any preparations of Cannabis (e.g., processed plant material). According to one embodiment, the natural cannabidiol may be extracted from Cannabis sativa or one of its preparations, e.g., marijuana, hashish, etc. According to one embodiment, the natural cannabidiol can be extracted from Cannabis using methods such as described, for example, in U.S. Pat. No. 6,403,123, and in Gaoni and Mechoulam [J Chem Soc 93:217-224 (1971)], both incorporated herein by reference.

The cannabidiol may also be a synthetic cannabidiol or a derivative thereof which can be generated using methods such as those described, for example and without being limited thereto, in US 2003/166727, and incorporated herein by reference.

In some embodiments, the composition comprises a CBD derivative. The term “CBD derivative” as used herein means a CBD derivative having an anti-inflammatory effect, or an analgesic effect, or having an ameliorating effect on inflammatory disease, disorder or conditions; or alternatively, a CBD derivative that binds to CB(1) and/or CB(2) cannabinoid receptors.

In some embodiments, a CBD derivative is selected from (−)-7-hydroxy-CBD, which is known from WO 2015/198077 to reduce triglyceride levels and treat fatty liver; (−)-CBD-7-oic acid, which is known from Haj 2015 to have an anti-inflammatory effect; and the dimethylheptvl (DMH) homolog of CBD, which is known to have an anti-inflammatory effect (Ben-Shabat 2006; Juknat 2016), and the corresponding compounds in the enantiomeric (+)- CBD series.

In some embodiments, a CBD derivative is characterized by a structure, wherein at least one of the hydroxyl substituent groups is converted to a stable form thereof. In some embodiments, a CBD derivative is cannabinol comprising a quinone ring. In one embodiment, a CBD derivative is an endocannabinoid derivative. In another embodiment, the pentyl group on the phenyl ring of the CBD is replaced with any straight-chain or branched alkyl group selected from (C1-C18)alkyl, optionally substituted.

In some embodiments the CBD is prepared from a cannabis extract. In some embodiments the term “CBD or a derivative thereof” refers to between 80% and 99% pure CBD. In some embodiments the term “CBD or a derivative thereof refers to between 90% and 99% pure CBD. In some embodiments the term “CBD or a derivative thereof” refers to between 93% and 99% pure CBD. In some embodiments the term “CBD or a derivative thereof refers to between 95% and 99% pure CBD. In some embodiments the term “CBD or a derivative thereof refers to between 95% and 97% pure CBD. In some embodiments the term “CBD or a derivative thereof” refers to about 97% pure CBD. All % hereinabove are weight %.

In some embodiments, the CBD or a derivative thereof is substantially and/or essentially devoid of tetrahydrocannabinol (THC). In some embodiments, a composition of the invention, as described herein, is substantially and/or essentially devoid of THC. In one embodiment, substantially and/or essentially devoid of THC means less than 9%» by weight THC. In one embodiment, substantially and/or essentially devoid of THC is less than 7% by weight THC. In one embodiment, substantially and/or essentially devoid of THC is less than 5% by weight THC. In one embodiment, substantially and/or essentially devoid of THC is less than 3% by weight THC. In one embodiment, substantially and/or essentially devoid of THC is less than 1% by weight THC. In one embodiment, substantially and/or essentially devoid of THC is less than 0.5% by weight THC. In one embodiment, substantially and/or essentially devoid of THC is less than 0.3% by weight THC. In one embodiment, substantially and/or essentially devoid of THC is less than 0.1% by weight THC.

In some embodiments, the CBD is synthetically prepared.

### Terpenes

Terpenes are a class of compounds with the formula (C₅H₈)n. Comprising more than 30,000 compounds, these unsaturated hydrocarbons are produced predominantly by plants, particularly conifers. Terpenes are further classified by the number of carbons: monoterpenes (C10), sesquiterpenes (C15), diterpenes (C20), etc. Terpenes useful in the invention include terpenes having 10 carbon atoms (C10) to 25 carbon atoms (C25). Preferred terpenes useful in the invention are terpenes having 10 carbon atoms or 15 carbon atoms. Preferred terpene mixtures are those having a predominant amount, i.e., more than 50% by weight, of C10 and C15 terpenes. Additionally preferred mixtures are those having a predominant amount, i.e., more than 50% by weight, of C10 terpenes.

The CBD formulation comprises at least eight terpenes, wherein the eight terpenes comprise linalool, myrcene, phytol, limonene, α-terpinene, α-terpineol, α-pinene, and β-caryophyllene. The weight ratio of linalool to myrcene is about 1.0:1.0, myrcene to phytol is about 1.0:1.0, phytol to limonene is about 1.0:1.0, limonene to α-terpinene is about 1.0:1.0, α-terpinene to α-terpineol is about 1.0:1.0, α-terpineol to α-pinene is about 1.0:1.0, and α-pinene to β-caryophyllene is about 1.0:1.0. In other embodiments, the weight ratio of linalool to myrcene ranges from 1.0:2.0 to 2.0:1.0, myrcene to phytol ranges from 1.0:2.0 to 2.0:1.0, phytol to limonene ranges from 1.0:2.0 to 2.0:1.0, limonene to α-terpinene ranges from 1.0:2.0 to 2.0:1.0, α-terpinene to α-terpineol ranges from 1.0:2.0 to 2.0:1.0, α-terpineol to α-pinene ranges from 1.0:2.0 to 2.0:1.0, and α-pinene to β-caryophyllene ranges from 1.0:2.0 to 2.0:1.0. In additional embodiments of the forgoing, the weight ratio of linalool to myrcene ranges from 1:0.9 to 0.9 to 1, myrcene to phytol ranges from 1:0.9 to 0.9 to 1, phytol to limonene ranges from 1:0.9 to 0.9 to 1, limonene to α-terpinene ranges from 1:0.9 to 0.9 to 1, α-terpinene to α-terpineol ranges from 1:0.9 to 0.9 to 1, α-terpineol to α-pinene ranges from 1:0.9 to 0.9 to 1, and α-pinene to β-caryophyllene ranges from 1:0.9 to 0.9 to 1. In additional embodiments of the forgoing, the weight ratio of linalool to myrcene ranges from 1:0.8 to 0.8 to 1, myrcene to phytol ranges from 1:0.8 to 0.8 to 1, phytol to limonene ranges from 1:0.8 to 0.8 to 1, limonene to α-terpinene ranges from 1:0.8 to 0.8 to 1, α-terpinene to α-terpineol ranges from 1:0.8 to 0.8 to 1, α-terpineol to α-pinene ranges from 1:0.8 to 0.8 to 1, and α-pinene to β-caryophyllene ranges from 1:0.8 to 0.8 to 1. In a specific embodiment, the weight ratio of linalool to myrcene ranges from 1.2:1.0 to 1.0:1.2, myrcene to phytol ranges from 1.2:1.0 to 1.0:1.2, phytol to limonene ranges from 1.2:1.0 to 1.0:1.2, limonene to α-terpinene ranges from 1.2:1.0 to 1.0:1.2, α-terpinene to α-terpineol ranges from 1.2:1.0 to 1.0:1.2, α-terpineol to α-pinene ranges from 1.2:1.0 to 1.0:1.2, and α-pinene to β-caryophyllene ranges from 1.2:1.0 to 1.0:1.2. In a specific embodiment, the weight ratio of linalool to myrcene ranges from 1.1:1.0 to 1.0:1.1, myrcene to phytol ranges from 1.1:1.0 to 1:0:1.1, phytol to limonene ranges from 1.1:1.0 to 1.0:1.1, limonene to α-terpinene ranges from 1.1:1.0 to 1.0:1.1, α-terpinene to α-terpineol ranges from 1.1:1.0 to 1.0:1.1, α-terpineol to α-pinene ranges from 1.1:1.0 to 1.0:1.1, and α-pinene to β-caryophyllene ranges from 1.1:1.0 to 1.0:1.1.

In one embodiment, the eight terpenes comprise linalool, myrcene, phytol, limonene, α-terpinene, α-terpineol, α-pinene, and β-caryophyllene are administered in a dose-response or dose-dependent manner to identify the precise concentration of each terpene that elicited the maximal response. The dose-response manner describes the magnitude of the response of the subject, as a function of exposure (or doses) to a terpene after a certain exposure time. Dose-response relationships can be described by dose-response curves.

Linalool is a monoterpenoid that is octa-1,6-diene substituted by methyl groups at positions 3 and 7 and a hydroxy group at position 3. It has been isolated from plants like Ocimum canum. Linalool refers to two enantiomers of a naturally occurring terpene alcohol found in many flowers and spice plants. Linalool has multiple commercial applications, the majority of which are based on its pleasant scent. A colorless oil, linalool is classified as an acyclic monoterpenoid.

Myrcene, or β-myrcene, is an alkene natural hydrocarbon (7-methyl-3-methylene-1,6-octadiene). Myrcene is more precisely classified as a monoterpene. Monoterpenes are dimers of isoprenoid precursors, and myrcene is the primary component of the essential oil of the South African Adenandra villosa, Myrcene is also found in bay, cannabis, and hops. In one embodiment, α-Myrcene is the structural isomer 2-methyl-6-methylene-1,7-octadiene. Myrcene possesses analgesic, antioxidant, anti depressant, anti-inflammatory, and muscle relaxing effects. Myrcene affects the permeability of the cell membranes, allowing more CBD and/or terpenese to reach nerve cells.

Phytol (florasol, phytosol) is an acyclic diterpene alcohol (2E,7R,11R)-3,7,11,15-Tetramethylhexadec-2-en-1-ol or a hexadec-2-en-1-ol substituted by methyl groups at positions 3, 7, 11 and 15. Diterpenes are composed of four isoprene units and have the molecular formula C₂₀H₃₂. Examples of diterpenes and diterpenoids are cafestol, kahweol, cembrene and taxadiene (precursor of taxol).

Limonene is a colorless liquid aliphatic hydrocarbon classified as a cyclic monoterpene, and is 1-methyl-4-prop-1-en-2-ylcyclohexene. The D-isomer, occurring more commonly in nature as the fragrance of oranges, is a flavoring agent in food manufacturing. d-limonene ((+)-limonene), which is the (R)-enantiomer. Racemic limonene is known as dipentene. Limonene can be an anti-depressant and can synergistically promote the absorption of other terpenes by quickly penetrating cell membranes. The result can be increased systolic blood pressure.

Alpha-terpinene (α-terpinene) is one of three isomeric monoterpenes differing in the positions of their two double bonds (beta- and gamma-terpinene being the others). In alpha-terpinene the double bonds are at the 1- and 3-positions of the p-menthane skeleton and is known as 1-methyl-4-propan-2-ylcyclohexa-1,3-diene. It has a role as a volatile oil component and a plant metabolite. It is a monoterpene and a cyclohexadiene. α-Terpinene has been isolated from cardamom and marjoram oils, and from other natural sources.

Alpha-terpineol (α-terpineol) is a terpineol that is propan-2-ol substituted by a 4-methylcyclohex-3-en-1-yl group at position 2. Alpha-terpineol is known as 2-(4-methylcyclohex-3-en-1-yl)propan-2-ol and has a role as a plant metabolite. Terpineol is any of four isomeric monoterpenoids. Terpenoids are terpene that are modified by the addition of a functional group, in this case, an alcohol. Terpineols have been isolated from a variety of sources such as cardamom, cajuput oil, pine oil, and petitgrain oil. Four isomers exist: α-, β-, γ-terpineol, and terpinen-4-ol. β- and γ-terpineol differ only by the location of the double bond. Terpineol is usually a mixture of these isomers with α-terpineol as the major constituent.

Alpha-Pinene (α-pinene) is an organic compound of the terpene class, one of two isomers of pinene and known as 2,6,6-trimethylbicyclo[3.1.1]hept-2-ene. It is an alkene and it contains a reactive four-membered ring, or bicyclo[3.1.1]hept-2-ene substituted by methyl groups at positions 2, 6 and 6 respectively. It is found in the oils of many species of many coniferous trees, notably the pine. It is also found in the essential oil of rosemary and Satureja myrtifolia. Both enantiomers are known in nature; (1S,5S)- or (−)-α-pinene is more common in European pines, whereas the (1R,5R)- or (+)-α-isomer is more common in North America. The racemic mixture is present in some oils such as eucalyptus oil and orange peel oil.

Beta-caryophyllene (β-caryophyllene) is a natural bicyclic sesquiterpene that is a constituent of many essential oils, especially clove oil, the oil from the stems and flowers of Syzygium aromaticum, the essential oil of Cannabis sativa, rosemary, and hops. β-caryophyllene is usually found as a mixture with isocaryophyllene (the cis double bond isomer) and α-humulene (obsolete name: α-caryophyllene), a ring-opened isomer. Caryophyllene is notable for having a cyclobutane ring, as well as a trans-double bond in a 9-membered ring.

### DESCRIPTION OF EMBODIMENTS

Generally speaking, the CBD formulation is composed of highly purified (>99.9%) hemp-derived cannabidiol (CBD) and terpenes as a sleep aid for people with insomnia and other sleep disorders. In one embodiment, the CBD formulation contains no detectable Δ-9-tetrahydrocannabinol (Δ-9-THC). This CBD formulation includes a plurality of terpenes acting synergistically with CBD and the CBD formulation use for the treatment of disease. In one embodiment, the CBD formulation comprises about 150 mg of CBD and about 0.5 mg each of the terpenes linalool, myrcene, phytol, limonene, α-terpinene, α-terpineol, α-pinene, and β-caryophyllene, and their use for the treatment of insomnia. In one embodiment, an effective dose for treating insomnia and other sleep disorders is about 300 mg CBD and about 1.0 mg each of the terpenes linalool, myrcene, phytol, limonene, α-terpinene, α-terpineol, α-pinene, and β-caryophyllene. In other embodiments, the effective dose for each individual with insomnia or other sleep disorder may vary, as indicated below.

In one embodiment, the CBD formulation comprises about 150 mg of CBD and about 0.5 mg each of the terpenes linalool, myrcene, phytol, limonene, α-terpinene, α-terpineol, α-pinene, and β-caryophyllene, and their synergistic use with CBD for the treatment of anxiety. In one embodiment, an effective dose for treating anxiety is about 300 mg CBD and about 1.0 mg each of the terpenes linalool, myrcene, phytol, limonene, α-terpinene, α-terpineol, α-pinene, and β-caryophyllene. In other embodiments, the effective dose for each individual with anxiety may vary, as indicated below.

In one embodiment, the CBD formulation comprises about 150 mg of CBD and about 0.5 mg each of the terpenes linalool, myrcene, phytol, limonene, α-terpinene, α-terpineol, α-pinene, and β-caryophyllene, and their synergistic use with CBD for the treatment of depression. In one embodiment, an effective dose for treating depression is about 300 mg CBD and about 1.0 mg each of the terpenes linalool, myrcene, phytol, limonene, α-terpinene, α-terpineol, α-pinene, and β-caryophyllene. In other embodiment, the effective dose for each individual with depression may vary, as indicated below.

In some embodiments, a CBD formulation provided herein may comprise between about 50 mg to about 4000 mg of CBD per effective dose, alternatively 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 2250 mg, 2500 mg, 2750 mg, 3000 mg, 3500 mg, or 4000 mg of CBD per effective dose.

In some embodiments, a CBD formulation provided herein may include about 0.1 mg to 5.0 mg of linalool per effective dose. For example, a conjugated formulation provided herein may comprise 0.1 mg to 3.0 mg, 0.5 mg to 3.0 mg, 1 mg to 3.0 mg, 0.1 mg to 2.5 mg, 0.5 mg to 2.5 mg, 1 mg to 2.5 mg, 0.1 mg to 2.0 mg, 0.5 mg to 2.0 mg, 1 mg to 2.0 mg, 0.1 mg to 1.5 mg, 0.5 mg to 1.5 mg, 1 mg to 1.5 mg/ml, 0.1 mg to 1.0 mg, 0.5 mg to 1.0 mg, 1 mg to 10 mg, 0.1 mg to 5.0 mg, 0.5 mg to 5.0 mg, 1 mg to 5.0 mg, 0.1 mg to 2 mg, 0.5 mg to 2 mg, 1 mg to 2 mg, 2 mg to 4.0 mg, 2 mg to 3.0 mg, 2 mg to 2.5 mg, 2 mg to 5.0 mg, 2 mg to 4.5 mg, 2 mg to 2.5 mg, or between 2 mg to 5 mg of linalool per effective dose. Alternatively, the dose may comprise 10 mg to about 100 mg of linalool per effective dose.

In some embodiments, a CBD formulation provided herein may include about 0.1 mg to 5.0 mg of myrcene per effective dose. For example, a conjugated formulation provided herein may comprise 0.1 mg to 3.0 mg, 0.5 mg to 3.0 mg, 1 mg to 3.0 mg, 0.1 mg to 2.5 mg, 0.5 mg to 2.5 mg, 1 mg to 2.5 mg, 0.1 mg to 2.0 mg, 0.5 mg to 2.0 mg, 1 mg to 2.0 mg, 0.1 mg to 1.5 mg, 0.5 mg to 1.5 mg, 1 mg to 1.5 mg/ml, 0.1 mg to 1.0 mg, 0.5 mg to 1.0 mg, 1 mg to 10 mg, 0.1 mg to 5.0 mg, 0.5 mg to 5.0 mg, 1 mg to 5.0 mg, 0.1 mg to 2 mg, 0.5 mg to 2 mg, 1 mg to 2 mg, 2 mg to 4.0 mg, 2 mg to 3.0 mg, 2 mg to 2.5 mg, 2 mg to 5.0 mg, 2 mg to 4.5 mg, 2 mg to 2.5 mg, or between 2 mg to 5 mg of myrcene per effective dose. Alternatively, the dose may comprise 10 mg to about 100 mg of myrcene per effective dose.

In some embodiments, a CBD formulation provided herein may include about 0.1 mg to 5.0 mg of phytol per effective dose. For example, a conjugated formulation provided herein may comprise 0.1 mg to 3.0 mg, 0.5 mg to 3.0 mg, 1 mg to 3.0 mg, 0.1 mg to 2.5 mg, 0.5 mg to 2.5 mg, 1 mg to 2.5 mg, 0.1 mg to 2.0 mg, 0.5 mg to 2.0 mg, 1 mg to 2.0 mg, 0.1 mg to 1.5 mg, 0.5 mg to 1.5 mg, 1 mg to 1.5 mg/ml, 0.1 mg to 1.0 mg, 0.5 mg to 1.0 mg, 1 mg to 10 mg, 0.1 mg to 5.0 mg, 0.5 mg to 5.0 mg, 1 mg to 5.0 mg, 0.1 mg to 2 mg, 0.5 mg to 2 mg, 1 mg to 2 mg, 2 mg to 4.0 mg, 2 mg to 3.0 mg, 2 mg to 2.5 mg, 2 mg to 5.0 mg, 2 mg to 4.5 mg, 2 mg to 2.5 mg, or between 2 mg to 5 mg of phytol per effective dose. Alternatively, the dose may comprise 10 mg to about 100 mg of phytol per effective dose.

In some embodiments, a CBD formulation provided herein may include about 0.1 mg to 5.0 mg of limonene per effective dose. For example, a conjugated formulation provided herein may comprise 0.1 mg to 3.0 mg, 0.5 mg to 3.0 mg, 1 mg to 3.0 mg, 0.1 mg to 2.5 mg, 0.5 mg to 2.5 mg, 1 mg to 2.5 mg, 0.1 mg to 2.0 mg, 0.5 mg to 2.0 mg, 1 mg to 2.0 mg, 0.1 mg to 1.5 mg, 0.5 mg to 1.5 mg, 1 mg to 1.5 mg/ml, 0.1 mg to 1.0 mg, 0.5 mg to 1.0 mg, 1 mg to 10 mg, 0.1 mg to 5.0 mg, 0.5 mg to 5.0 mg, 1 mg to 5.0 mg, 0.1 mg to 2 mg, 0.5 mg to 2 mg, 1 mg to 2 mg, 2 mg to 4.0 mg, 2 mg to 3.0 mg, 2 mg to 2.5 mg, 2 mg to 5.0 mg, 2 mg to 4.5 mg, 2 mg to 2.5 mg, or between 2 mg to 5 mg of limonene per effective dose. Alternatively, the dose may comprise 10 mg to about 100 mg of limonene per effective dose.

In some embodiments, a CBD formulation provided herein may include about 0.1 mg to 5.0 mg of α-terpinene per effective dose. For example, a conjugated formulation provided herein may comprise 0.1 mg to 3.0 mg, 0.5 mg to 3.0 mg, 1 mg to 3.0 mg, 0.1 mg to 2.5 mg, 0.5 mg to 2.5 mg, 1 mg to 2.5 mg, 0.1 mg to 2.0 mg, 0.5 mg to 2.0 mg, 1 mg to 2.0 mg, 0.1 mg to 1.5 mg, 0.5 mg to 1.5 mg, 1 mg to 1.5 mg/ml, 0.1 mg to 1.0 mg, 0.5 mg to 1.0 mg, 1 mg to 10 mg, 0.1 mg to 5.0 mg, 0.5 mg to 5.0 mg, 1 mg to 5.0 mg, 0.1 mg to 2 mg, 0.5 mg to 2 mg, 1 mg to 2 mg, 2 mg to 4.0 mg, 2 mg to 3.0 mg, 2 mg to 2.5 mg, 2 mg to 5.0 mg, 2 mg to 4.5 mg, 2 mg to 2.5 mg, or between 2 mg to 5 mg of α-terpinene per effective dose. Alternatively, the does may comprise 10 mg to about 100 mg of α-terpinene per effective dose.

In some embodiments, a CBD formulation provided herein may include about 0.1 mg to 5.0 mg of α-terpineol per effective dose. For example, a conjugated formulation provided herein may comprise 0.1 mg to 3.0 mg, 0.5 mg to 3.0 mg, 1 mg to 3.0 mg, 0.1 mg to 2.5 mg, 0.5 mg to 2.5 mg, 1 mg to 2.5 mg, 0.1 mg to 2.0 mg, 0.5 mg to 2.0 mg, 1 mg to 2.0 mg, 0.1 mg to 1.5 mg, 0.5 mg to 1.5 mg, 1 mg to 1.5 mg/ml, 0.1 mg to 1.0 mg, 0.5 mg to 1.0 mg, 1 mg to 10 mg, 0.1 mg to 5.0 mg, 0.5 mg to 5.0 mg, 1 mg to 5.0 mg, 0.1 mg to 2 mg, 0.5 mg to 2 mg, 1 mg to 2 mg, 2 mg to 4.0 mg, 2 mg to 3.0 mg, 2 mg to 2.5 mg, 2 mg to 5.0 mg, 2 mg to 4.5 mg, 2 mg to 2.5 mg, or between 2 mg to 5 mg of α-terpineol per effective dose. Alternatively, the dose may comprise 10 mg to about 100 mg of α-terpineol per effective dose.

In some embodiments, a CBD formulation provided herein may include about 0.1 mg to 5.0 mg of α-pinene per effective dose. For example, a conjugated formulation provided herein may comprise 0.1 mg to 3.0 mg, 0.5 mg to 3.0 mg, 1 mg to 3.0 mg, 0.1 mg to 2.5 mg, 0.5 mg to 2.5 mg, 1 mg to 2.5 mg, 0.1 mg to 2.0 mg, 0.5 mg to 2.0 mg, 1 mg to 2.0 mg, 0.1 mg to 1.5 mg, 0.5 mg to 1.5 mg, 1 mg to 1.5 mg/ml, 0.1 mg to 1.0 mg, 0.5 mg to 1.0 mg, 1 mg to 10 mg, 0.1 mg to 5.0 mg, 0.5 mg to 5.0 mg, 1 mg to 5.0 mg, 0.1 mg to 2 mg, 0.5 mg to 2 mg, 1 mg to 2 mg, 2 mg to 4.0 mg, 2 mg to 3.0 mg, 2 mg to 2.5 mg, 2 mg to 5.0 mg, 2 mg to 4.5 mg, 2 mg to 2.5 mg, or between 2 mg to 5 mg of α-pinene per effective dose. Alternatively, the dose may comprise 10 mg to about 100 mg of α-pinene per effective dose.

In some embodiments, a CBD formulation provided herein may include about 0.1 mg to 5.0 mg of beta-caryophyllene per effective dose. For example, a conjugated formulation provided herein may comprise 0.1 mg to 3.0 mg, 0.5 mg to 3.0 mg, 1 mg to 3.0 mg, 0.1 mg to 2.5 mg, 0.5 mg to 2.5 mg, 1 mg to 2.5 mg, 0.1 mg to 2.0 mg, 0.5 mg to 2.0 mg, 1 mg to 2.0 mg, 0.1 mg to 1.5 mg, 0.5 mg to 1.5 mg, 1 mg to 1.5 mg/ml, 0.1 mg to 1.0 mg, 0.5 mg to 1.0 mg, 1 mg to 10 mg, 0.1 mg to 5.0 mg, 0.5 mg to 5.0 mg, 1 mg to 5.0 mg, 0.1 mg to 2 mg, 0.5 mg to 2 mg, 1 mg to 2 mg, 2 mg to 4.0 mg, 2 mg to 3.0 mg, 2 mg to 2.5 mg, 2 mg to 5.0 mg, 2 mg to 4.5 mg, 2 mg to 2.5 mg, or between 2 mg to 5 mg of beta-caryophyllene per effective dose. Alternatively, the dose may comprise 10 mg to about 100 mg of beta-caryophyllene per effective dose.

### CBD and Terpenes Influences Sleep Physiology

The CBD formulation is tested on sleep physiology in a properly controlled, well-powered and rigorous study in humans.

The CBD formulation comprises a highly purified (>98% purity) forms of the terpenes linalool, myrcene, phytol, limonene, α-terpinene, α-terpineol, α-pinene, and β-caryophyllene [Food Grade (GRAS), Natural, Organic, non-GMO, dairy-free, gluten-free, vegan). Preclinical studies in animals have shown that these terpenes are sedating⁵⁻⁹, but their potential effects on sleep physiology in humans have never been established. Importantly, these terpenes are designated by the FDA as GRAS (Generally Regarded as Safe) for human oral consumption and have been used as food additives for decades.

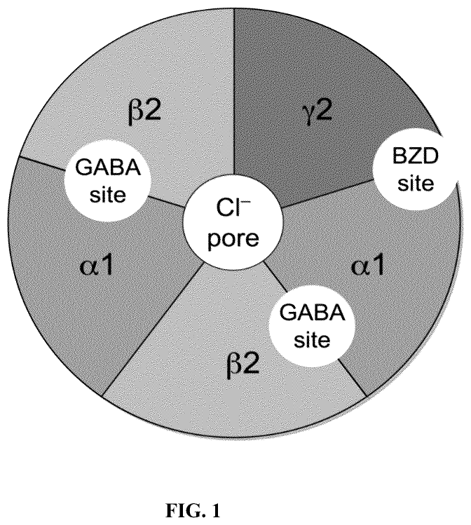

A number of published studies indicate that some of these terpenes (myrcene, α-pinene, phytol, terpinolene and α-terpineol) induce sedation by a similar mechanism of action in which they modulate the Gamma aminobutyric acid (GABA) neurotransmitter system (FIG. 1). The GABA_{A} receptor is an ionotropic receptor and ligand-gated ion channel. Its endogenous ligand is γ-aminobutyric acid (GABA), the major inhibitory neurotransmitter in the central nervous system that exerts its effects through reducing neuronal activity.

### Mechanism of GABAergic Terpenes GABA_{A} Sedative Activity

There are two receptors for GABA—the GABA_{A} receptor (an ion channel) and the GABA_{B} receptor (a G protein-coupled receptor). A class of clinically prescribed sedatives called benzodiazepines work through the GABA_{A} receptors. Benzodiazepines are widely used clinically to treat anxiety and insomnia¹⁰. Indeed, the benzodiazepines alprazolam (Xanax), clonazepam (Klonopin), diazepam (Valium), and lorazepam (Ativan) are among the most commonly prescribed drugs in the U.S. Zolpidem (Ambien) is a commonly prescribed insomnia drug which acts as a sedative and hypnotic. It also works by increasing GABA effects in the central nervous system by binding to GABA_{A} receptors at the same location as benzodiazepines. Other commonly prescribed drugs for insomnia, including Triazolam (Halcion) and Zaleplon (Sonata), also act by binding to GABA_{A} receptors at the same location as benzodiazepines. All of these sleep medications increase activation of the receptor by GABA, leading to an inhibition in neuronal activity, sedation and hypnosis.

Terpenes described above exert their effects through the same benzodiazepine binding site of GABA_{A} receptors. The pharmacologic profile of these terpenes is similar to that of benzodiazepines. Flumazenil, which is an antagonist of the benzodiazepine binding site, blocks the effects of these terpenes on sedation in vitro and in vivo in rodents^{8, 11,12}. Myrcene and α-pinene increased GABA_{A} receptor activity in cell experiments¹³. The effect of α-pinene in neurons is fully blocked by flumazenil¹³. In rodents, flumazenil blocked the sedative effects of lemongrass oil (which has high myrcene content)¹⁴, α-pinene¹¹, and phytol¹². α-terpineol can also increase GABA_{A} activity in cells¹⁵. Even though its pharmacology in rodents has not been characterized, it will be similar to the other GABAergic terpenes. Phytol may have an additional GABA-related mechanism through inhibiting SSADH, the enzyme that breaks down GABA¹⁶. Thus, it may be able to raise GABA levels in the brain in addition to increasing activation of the GABA_{A} receptor.

The terpene limonene also causes sedation in animals. For example, a study in rodents found that limonene reduced locomotor activity, induced sleep, and impaired motor coordination⁸. Another study showed that after one week of limonene dosing, brain GABA levels were increased, an effect that could be blocked by the GABA_{A} antagonist flumazenil¹⁷.

The terpene linalool also increases sleep in rodents¹⁸. Linalool does not bind to the GABA site or to the benzodiazepine site of the GABA_{A} receptor. Nonetheless, a 2014 study showed it could significantly augment GABA_{A} activity in cells¹³.

The sedative properties of the terpene β-caryophyllene are distinct from the GABAergic terpenes^{19,20}. In rodents, β-caryophyllene did not affect locomotor activity or motor coordination. However, it did mildly promote sleep^{19,20}. The mechanism of action of β-caryophyllene to induce sedation is not clear, but appears to be independent of the GABA neurotransmitter system.

In summary, preclinical data indicates that all of the terpenes in the CBD formulation (linalool, myrcene, phytol, limonene, α-terpinene, α-terpineol, α-pinene, and β-caryophyllene) induce sedation in animals, and that the majority of these terpenes cause sedation by activating the benzodiazepine site on the GABA_{A} receptor, the same site that most prescription insomnia drugs also act on. But most importantly, the effects of these terpenes on sedation and insomnia in humans has never been established.

### CBD Decreases Anxiety

Several clinical studies indicate that CBD can decrease acute anxiety²¹. Participants with social anxiety disorder and controls were blindly allocated to receive CBD or a placebo control before a simulation public speaking test. CBD resulted in significantly reduced anxiety, cognitive impairment and discomfort, and significantly decreased hyper-alertness in anticipatory speech²². THC is well established as having anxiogenic (anxiety-promoting) properties in clinical studies, and these effects can be blocked by CBD^{23,24}.

The only disadvantage of using the CBD formulation relates to potential safety concerns regarding its potential to cause liver damage. However, these concerns are all due to concomitant use of a small number of rarely used prescription drugs, which are contra-indicated with the CBD formulation. In terms of the liver damage issue, it should be easy to minimize the adverse effects of the CBD formulation by posting warning labels on packaging.

In one embodiment, optimization of specific terpene concentrations found in the CBD formulation might result in a sleep aid with superior efficacy to the CBD formulation. Therefore, alternative terpene concentrations are provided herein.

In one embodiment, the subject or consumer determines the precise dose that works for them. The consumer will be guided by a novel smartphone-based application with a sleep coach or doctor.

The major advantage of the CBD formulation is a novel sleep aid that increases REM and restorative sleep that has an excellent safety profile. The effects of the CBD formulation on boosting restorative sleep cannot be overstated. The vast majority of benzodiazepine-based prescription insomnia drugs function as sedatives/hypnotics, and have not been shown to have any effect on restorative sleep. They simply knock you out, and have a large number of serious adverse reactions that limits their utility. Moreover, based on the clinical data collected below, the CBD formulation is also beneficial for treating anxiety and or depression. The CBD formulation might also have utility for other indications including but not limited to the treatment of pain, autism, and other sleep related disorders.

### Dosage Form

In one embodiment, the CBD formulation is in the form of capsules that consumers can take orally. The CBD formulation is free from any contaminants, including foreign material, pesticides, bacterial pathogens, molds, residual solvents, heavy metals and THC. A certificate of analysis (COA) documented the analytical tests for CBD formulation.

The methods described herein may comprise administering daily, or every other day, or once a week, an effective dose of the CBD formulation. In an embodiment, the CBD formulation is administered daily for 1-2 days, 1-3 days, 1-12 days, 4-8 days, 8-12 days, 1-24 days, 4-24 days, 8-24 days, or 12-24 days, or more, or for another period of time according to the present invention.

The CBD formulation of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the CBD formulation and not deleterious to the user. In accordance with one embodiment, suitable nutraceutically acceptable carriers can include oils, plant-based oils, Medium-chain triglyceride (MCT) oil, coconut oil, palm kernel oil, Hemp seed oil, Olive oil, Avocado oil, and combinations thereof.

In certain embodiments, the dosage form is formulated as granules, pellets, micro particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, gummy, chocolate or cookie. In certain embodiments, the tablet or pellets are an immediate release or slow or controlled release dosage forms. In certain embodiment the tablet is enteric coated or is a melt or dissolved in the mouth or is muco-adhesive dosage form.

In certain embodiments, the unit dosage form which is a unit particles, such as tablet, capsule, granules, pellets, micro-particles and film, are enteric coated or coated with a colonic coat that protect the unit dose from being decomposed at the acidic gastric pH and swells in time manner of pH controlled manner or both, to release the CBD formulation at the distal intestine and may also release part of the cannabinoids in the intestines for systemic absorption and part of the cannabinoids at the colon for local colonic pharmacological effect.

In certain embodiments, the CBD formulation is formulated in a semi solid or liquid dosage form such as cream, lotion, ointment, dispersion, suspension, gel, foam, spray, syrup, liquid, eye drops, ear drops, enema or an oral dosage form or a topical dosage form or a local ophthalmic or optic or oral cavity or vaginal or rectal or uterine dosage form. Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

In certain embodiments, any one of the compositions described above, or any one of the dosage forms described above, is for use in a method of treating sleep symptoms or disorders.

Preferred dosage forms include, but are not limited to, any liquid or semi solid or solid dosage form. The CBD formulation may be formulated in a medicament by preparing a topical or mucosal or oral delivery system. The topical delivery system may be in form of eye drops, a suspension, ointment, cream, foam, spray, topical patch. The oral delivery system may be a tablet or capsule or soft capsule or sachet or granules or a syrup. The mucosal delivery system may be a gel, pessary, enema, douche, wash, foam, mucoadhesive gel or tablet for immediate or for slow or controlled release. The vehicle may comprise any acceptable solvent and inactive ingredients as well as preservatives anti-oxidants and coloring agents. The delivery form may be single dose or multiple dose as well as micro particle granulate nanoparticle microcapsule liposome micelle, and the like as known in the art of pharmaceutical, cosmetic, veterinary medicine and art of formulation. Further details of suitable dosage forms may be obtained from any standard reference work in this field, including, for example: Remington's Pharmaceutical Sciences, Mack Publishing Co, Easton, Pa, USA (1980).

Thus, in some embodiments of the present invention, the CBD formulation further comprises one or more excipients selected from the group consisting of solvents, stabilizers, suspending agents, emulsifiers, release modifying, targeting and viscosity agents and combinations thereof.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example: Summary Of Data From Clinical Studies With the CBD formulation

Two independent clinical studies were completed to determine the effects of the CBD formulation on the structure and function of sleep physiology. Each of these independent studies were performed as a double-blind, placebo-controlled, randomized, crossover investigational study to determine if the CBD formulation influences sleep physiology in humans.

Primary sleep physiology data was collected from study participants in a completely unbiased manner from a non-invasive sleep-tracking wristband, called “Whoop” (https://www.whoop.com), which electronically collects and transmits sleep data from study participants in the comfort of their own beds. The wristband collects hundreds of data points per second from a 3-axis accelerometer, 3-axis gyroscope, and heart rate sensor. The wristband can accurately measure latency to fall asleep, total sleep time, sleep fragmentation, as well as the time spent in each sleep stage [Light, Slow Wave (Deep), Rapid Eye Movement (REM), and Awake]. The wristband also collects data using photoplethysmography (PPG), a technique that involves measuring blood flow by assessing superficial changes in blood volume. Heart rate, heart rate variability, and respiratory rate, can all be derived from PPG data, and all of these metrics are used in Whoop's sleep detection and staging algorithms. Two recent publications, published independently from Whoop, show that data on sleep stages collected from the Whoop device are highly accurate and correlate well with polysomnography (PSG), the gold-standard of sleep tracking used in clinical studies conducted in sleep clinics^{25,26}.

### Summary of Data Study 1

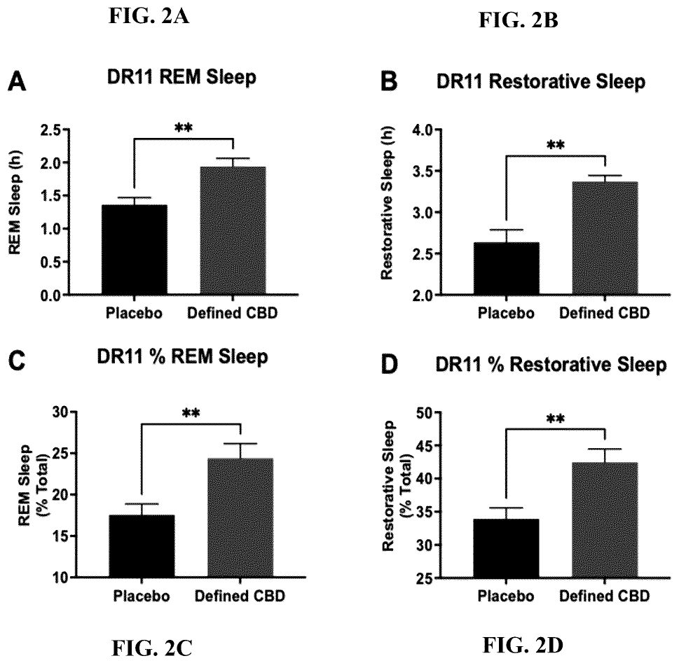

In the first study, participants (N=23) were randomized to take a placebo control or the CBD formulation stated as Defined CBD in the Figures (˜300 mg CBD, ˜8 mg terpenes including linalool, myrcene, phytol, limonene, α-terpinene, α-terpineol, α-pinene, and β-caryophyllene) once daily, one hour before bedtime, for seven consecutive nights. An example of one participant's data is shown in FIG. 2A-2D. Participant DR11 showed a significant increase in REM sleep of about 0.5 hours, restorative sleep of about 0.9 hours (the combination of REM and deep sleep), and the percentage of time spent in REM sleep of about 7% and restorative sleep of about 9% (FIGS. 2A-2D). Indeed, on average, over seven days of treatment, participant DR11 benefitted from nearly an extra hour of restorative sleep each night relative to the placebo control (FIG. 2B). Furthermore, participant DR11 was also able to perceive an improvement in their sleep quality and duration relative to the placebo control when they responded to a clinically validated sleep survey at the end of the treatment periods (as shown in Table 1).

TABLE 1

| | Improved | Decreased | Lengthened |
|---|---|---|---|
| DR11 | Sleep | Sleep Onset | Sleep Duration |
| Placebo | No | No | No |
| CBD formulation | Yes | No | Yes |
| CBD Alone | No | No | No |

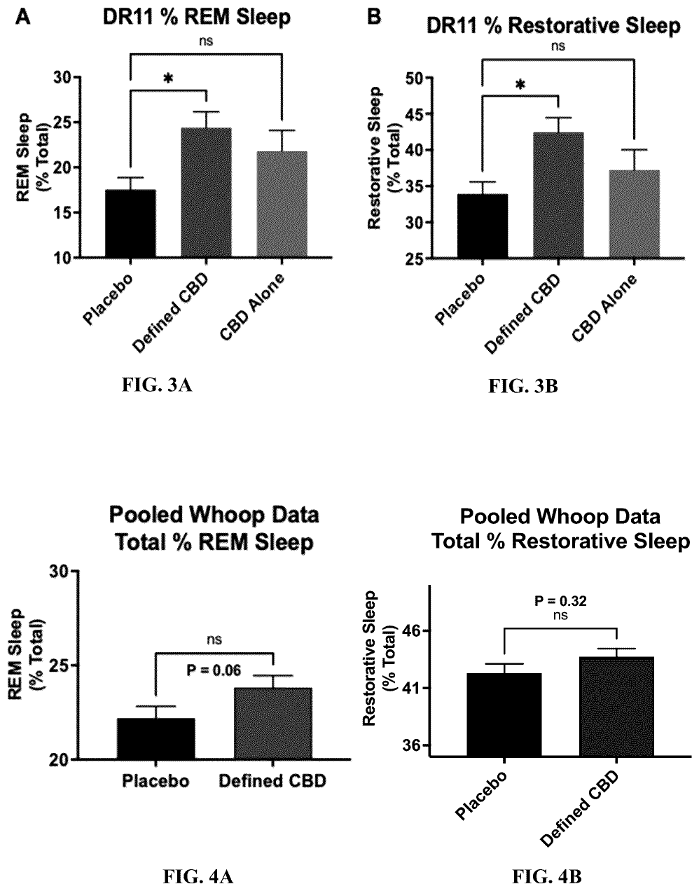

In some study participants, including DR11, while the CBD formulation significantly increased the percentage of time spent in REM sleep about 7% and restorative sleep of about 9%, CBD alone appeared to only partially increase the percentage of REM sleep of about 4% and restorative sleep of about 2%, but these effects were not statistically significant (FIGS. 3A-3B). This data suggests that the terpenes included in the CBD formulation synergize with CBD to contribute to its sleep benefits.

The analysis of the pooled data from all of the participants (N=23) from the first clinical study indicated that the CBD formulation showed a trend to increase the percentage of REM of about 2% (FIG. 4A) and restorative sleep of about 1% (as shown FIG. 4B).

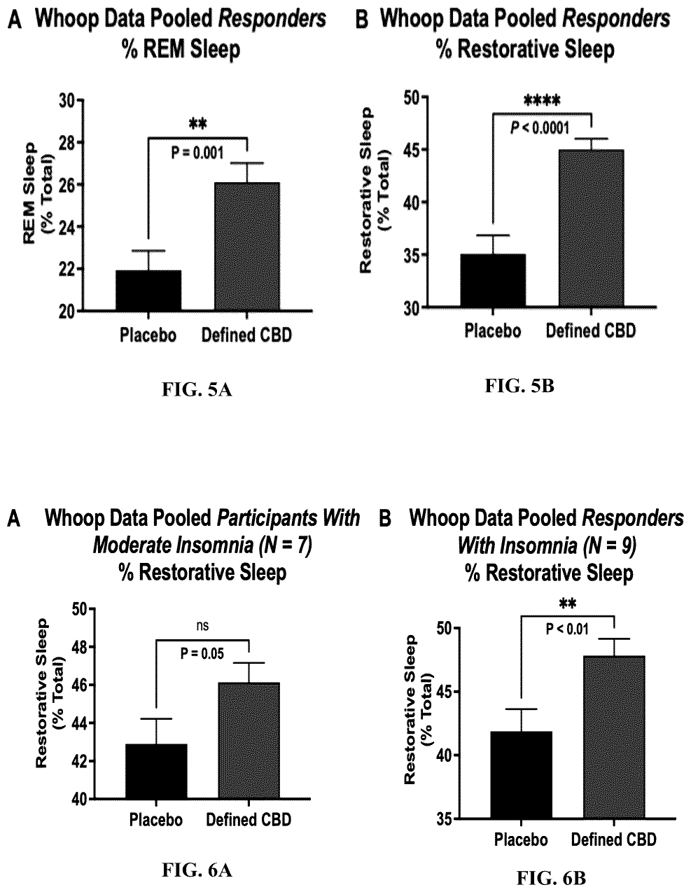

Analysis of the pooled data from the first clinical study indicated that participants could be stratified into “responders” and “non-responders”. Pooled data from “responders” showed a highly significant increase in the percentage of REM of about 4% and restorative sleep of about 10% in study participants who received the CBD formulation relative to the placebo control (FIGS. 5A-5B). Indeed, when only “responders” were analyzed, they exhibited a ˜10% increase in restorative sleep when treated with CBD formulation (FIG. 5B).

After more careful scrutiny, the participants who responded to treatment with the CBD formulation, 9/11 exhibited insomnia or subthreshold insomnia as defined by a clinically validated insomnia severity index [the Pittsburgh Sleep Quality Index (PSQI)]²⁷ that was completed prior to the clinical study being conducted. After taking the CBD formulation, participants who scored as having moderate insomnia in the PSQI exhibited an increase in the percentage of restorative sleep of about 3% that narrowly missed statistical significance, and a significant increase in the percentage of restorative sleep of about 6% relative to the placebo control (FIGS. 6A-6B).

At the end of each treatment period in the clinical studies, participants completed a modified version of a clinically validated questionnaire entitled the Participant Global Impression (PGI)²⁸. PGI assessments are based on the participant's global perception of the effects of treatment on sleep during each treatment period, as compared to their sleep during the baseline period of the study. Using the PGI assessment, participants perceived that they slept better after taking the CBD formulation with a score of 1.4 relative to the placebo control with a score of 1.6, although this effect did not achieve statistical significance (FIG. 7). Nevertheless, based on post-hoc power analysis performed, this effect would achieve statistical significance with a larger sample number.

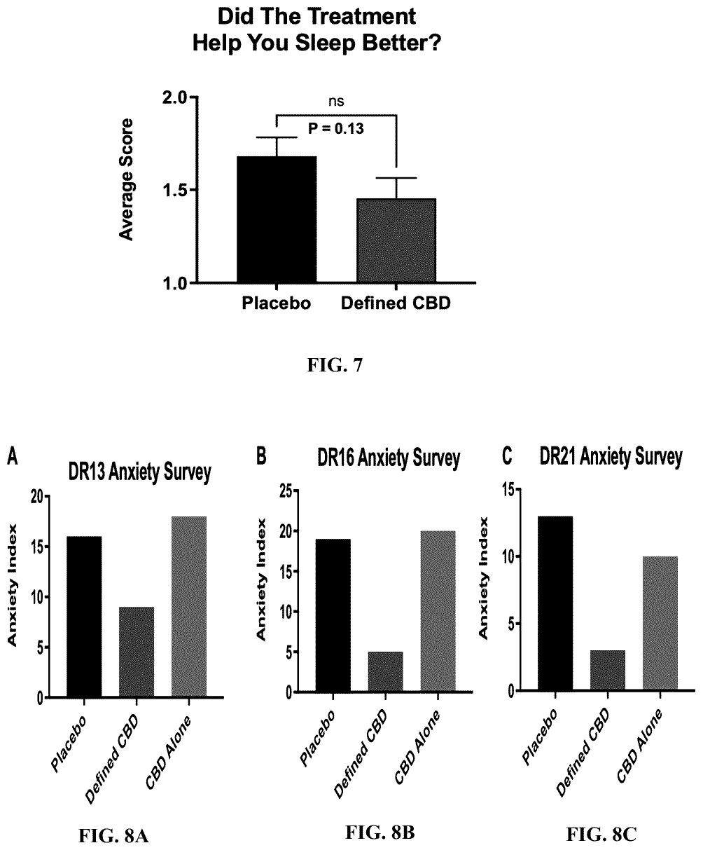

To measure the effects of the CBD formulation on anxiety, study participants completed a modified version of the Hamilton Anxiety Rating Scale, one of the first rating scales developed to measure the severity of anxiety symptoms that is widely used in clinical and research settings²⁹. This scale consists of 13 items, each defined by a series of symptoms, and measures both psychic anxiety (mental agitation and psychological distress) and somatic anxiety (physical complaints related to anxiety). Each item is scored on a scale of 0 (not present) to 4 (severe), with a total score range of 0-53, where <14 indicates mild severity, 15-21 mild to moderate severity, and 22-27 moderate to severe. Based on this clinically-validated anxiety rating scale, some study participants showed a decrease in anxiety with a score of 8, 5, and 3 after taking the CBD formulation, as shown in the participants' data in FIGS. 8A-8C.

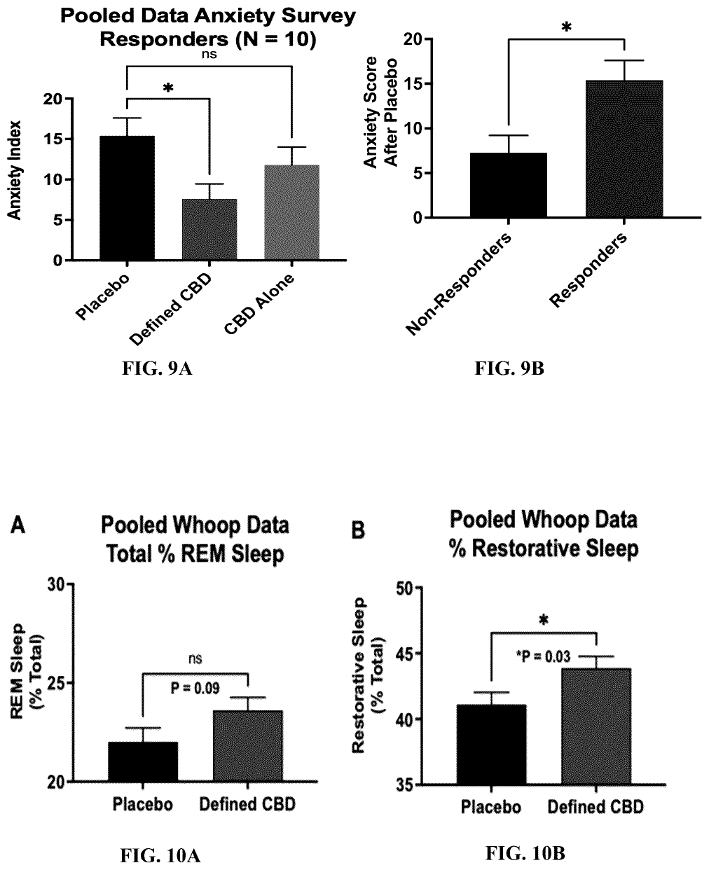

Analysis of the pooled data from the first clinical study showed no significant effect of the CBD formulation on anxiety relative to the placebo control. However, as with the sleep physiology data, the data could be stratified into “responders” and “non-responders”. Data from “responders” showed that the CBD formulation significantly lowered anxiety with a score of 6 relative to the placebo control with a score of 15 or CBD alone with a score of 11 (FIG. 9A). The study participants were not asked to take the Hamilton Anxiety Rating Scale prior to the study, it is not clear if those who most benefitted most from the CBD formulation were those who had the highest baseline anxiety levels. However, the average anxiety level of “responders” who took placebo was significantly higher than “non-responders”, suggesting that similar to the effects of the CBD formulation and insomnia levels on restorative sleep, participants who exhibited higher baseline anxiety levels might most benefit from the CBD formulation (as shown in FIG. 9B).

### Summary of Data from Study 2

After the successful completion of the first clinical study, the first clinical study results were attempted to be replicated it in an independent cohort of participants. As data from the first clinical study indicated that participants with insomnia most benefitted from treatment with the CBD formulation, for the second clinical study, only those who exhibited moderate or severe insomnia as defined by a clinically validated insomnia severity index (the PSQI)²⁷ were enrolled.

In the second study, participants (N=13) were randomized to take a placebo control or the CBD formulation (˜300 mg CBD, ˜8 mg terpenes including linalool, myrcene, phytol, limonene, α-terpinene, α-terpineol, α-pinene, and β-caryophyllene) once daily, one hour before bedtime, for at least four nights per week, for a total treatment period of three weeks.

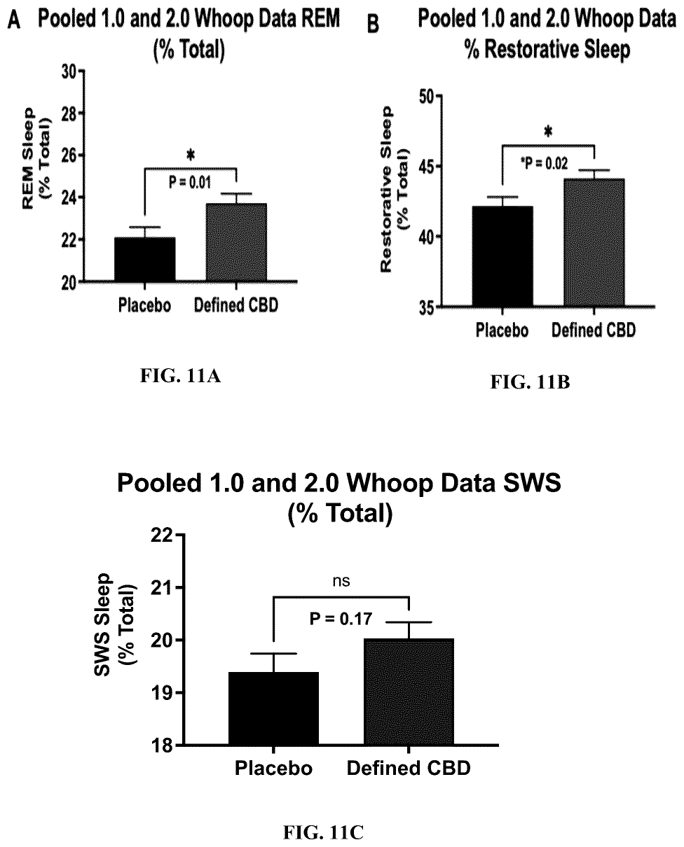

In pooled data from the second study, the CBD formulation increased the percentage of REM sleep by about 2% and restorative sleep by about 3% in participants treated with the CBD formulation relative to the placebo control, and restorative sleep was increased by about 3% and reached statistical significance, as shown in FIGS. 10A-10B. Similar to the first clinical study, this data represents an encouraging replication of the first clinical study in a completely independent patient cohort. Moreover, based on post-hoc power analysis that was performed, the effects of the CBD formulation on REM sleep will achieve statistical significance with a larger sample number.

The two clinical studies were performed in a decentralized manner in which study participants self-administered treatments and tracked their sleep data in the comfort of their own homes. The effects of the CBD formulation on sleep physiology were quantified when the data from the two clinical studies were combined. The CBD formulation significantly increased the percentage of time study participants spent in REM by about 2% and restorative sleep by about 2% in the combined data set, as shown in FIGS. 11A-11B. The effects of the CBD formulation on deep sleep in the combined data narrowly missed statistical significance but the CBD formulation increased deep sleep by about 2%, as shown FIG. 11C.

As before, based on power analysis performed, the effects of the CBD formulation on deep sleep will achieve statistical significance with a larger sample number. Indeed, the power analysis indicates that in a larger sample number, the magnitude of the statistical significance of the findings will likely increase and an additional final clinical study in 150 participants.

### Conclusions and Clinical Significance Of Research Findings

The clinical data with the CBD formulation indicates that it significantly increases the percentage of time people spend in REM and restorative (deep plus REM sleep), and that these effects are most pronounced in people suffering from insomnia. Importantly, the effects of the CBD formulation were replicated on these sleep physiology parameters in two independent patient cohorts. Furthermore, participants who took the CBD formulation could also perceive a benefit of the treatment on their sleep quality.

REM and restorative sleep appear to be critically important for good brain health, sand their absence in human populations is often associated with chronic ailments and diseases. For example, REM sleep has an essential role in the process of learning and memory in the hippocampus, and disruption of REM sleep can lead to cognitive impairment and other manifestations³⁰. During sleep, the brain is able to repair and grow cells, tissue, and nerves that regenerate and boost the hormone and immune system. Along with good nutrition, exercise and stress reduction, restorative sleep is vital for your optimal physical, mental, and emotional health. Insufficient restorative sleep increases the risk of disorders, such as high blood pressure, diabetes, obesity, stroke and depression. It's also associated with cognitive decline and Alzheimer's disease.

The CBD formulation is composed CBD and terpenes that have shown significantly increases REM and restorative sleep in people with insomnia, and these people could correctly perceive the benefit of this treatment (even though they were blinded to the treatments). In addition to its documented efficacy, one of the other major advantages of the CBD formulation as a potential sleep aid is its outstanding safety profile, especially relative to commonly prescribed sleep medications including Zolpidem (Ambien), which suffer from numerous and serious unwanted adverse reactions.

### Example 2: A Double-Blind, Placebo-Controlled, Randomized, Crossover Investigational Study to Determine if CBD Formulation, a Capsule with a Custom Formulation of Cannabidiol (CBD) and Terpenes, Influences Sleep Physiology

The novel CBD-based formulation in the form of a small orally administered capsule, will increase the percentage of time participants spend in deep (SWS) and REM sleep, as measured by an objective sleep-tracking device called “Whoop”. The research study, entitled Le Rêve 3.0, is a double-blind, placebo-controlled, randomized, crossover investigational study to determine if CBD formulation or labeled as CBD-Terpenes, a Custom-Formulated CBD Capsule, influences sleep physiology. This capsule contains no Δ-9-THC. As this study is only designed to evaluate the effects of CBD formulation on the structure and function of sleep physiology, the phase of the trial is not applicable.

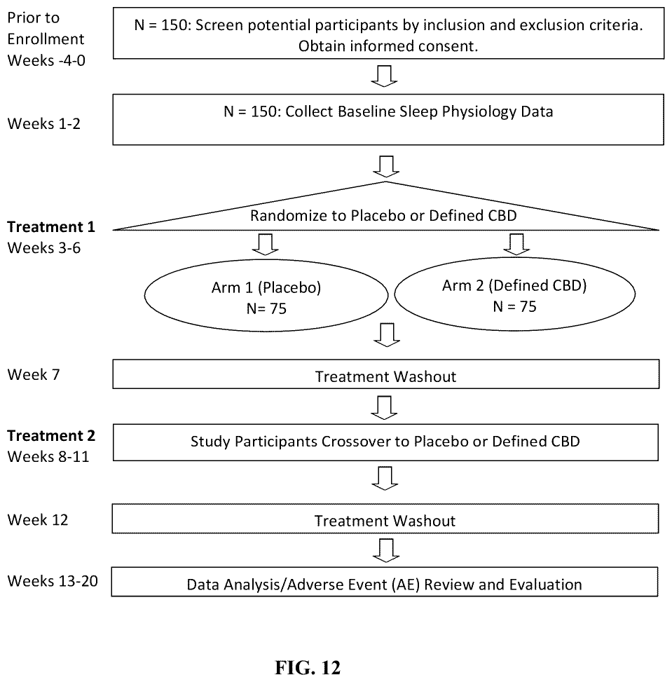

The Le Rêve 3.0 study will involve a cross-over design in which study participants will cycle through two independent treatments, each for four weeks (Treatment 1: Placebo; Treatment 2: CBD formulation). The placebo will look, smell and taste the same as the CBD formulation capsule. The study participants will initially be randomized with respect to the treatment arm, and every participant will cycle through both treatment arms. A double-blind design will be used in which the investigator and study participants will both be blinded to the treatments. Baseline data for each participant will be collected for two weeks prior to initiating treatments, as well as for a one-week washout period following each treatment arm. The entire study duration will be 12 weeks, as shown in FIG. 12.

Primary data for Le Rêve will be obtained in a completely unbiased manner from a non-invasive sleep-tracking wrist-worn device called “Whoop” (https://www.whoop.com), that electronically collects and transmits sleep data from study participants in the comfort of their own beds.

The wristband collects hundreds of data points per second from a 3-axis accelerometer, 3-axis gyroscope, and heart rate sensor. The wristband can accurately measure the latency to fall asleep, total sleep time, sleep fragmentation, as well as the time spent in each sleep stage [Light, Slow Wave (Deep), Rapid Eye Movement (REM), and Awake]. The device also collects data using photoplethysmography (PPG), a technique that involves measuring blood flow by assessing superficial changes in blood volume. Heart rate, heart rate variability and respiratory rate, can all be derived from PPG data, and all of these metrics are used in Whoop's sleep detection and staging algorithms. Importantly, two recent publications, published independently from Whoop, show that data on sleep stages collected from the Whoop device are highly accurate and correlate well with polysomnography (PSG), the gold-standard of sleep tracking used in clinical studies conducted in sleep clinics.

### Study Description

Insomnia is a disorder in which people have inadequate or poor-quality sleep due to a number of factors, such as difficulty falling asleep, waking up frequently during the night with difficulty returning to sleep, waking up too early in the morning, or having unrefreshing sleep. CBD formulation is a capsule composed of highly purified (>99.9% purity) hemp-derived CBD and terpenes (>98% purity). CBD formulation contains no Δ-9- tetrahydrocannabinol (Δ-9-THC). The research study, entitled Le Rêve 3.0, is designed to evaluate the efficacy of CBD formulation on sleep physiology.

Primary Objectives are to determine if CBD formulation influences objective measures of sleep physiology. Secondary Objectives are to determine if CBD formulation influences subjective measures of sleep physiology.

The Primary Endpoint is the percentage of sleep spent in deep [slow wave sleep (SWS)] and rapid eye movement (REM) sleep as determined using an objective wrist-worn sleep-tracking device called Whoop. Secondary Endpoints are the total sleep time, sleep latency, the number of sleep disturbances, time spent in each sleep stage, percentage of time spent in each sleep stage, and subjective measures of sleep physiology as collected via participant surveys.

Study Population: 150 study participants will be enrolled who display chronic insomnia defined as a self-reported difficulty initiating (latency to persistent sleep >30 min) and/or maintaining sleep (>30 min awake, or waking >30 min before desired waking time) on three or more nights per week for at least 3 months. Participants will also complete a brief clinically validated insomnia severity index, and only participants with an Insomnia Severity Index score >15 will be enrolled in Le Rêve 3.0. Males or females aged 25-70 years that are located in the United States that meet inclusion and exclusion criteria will be enrolled.

### Description of Study Interventions

Le Rêve 3.0 will be a decentralized study completed by participants in the comfort of their own homes. Recruitment, enrollment, informed consent, and distribution of study materials to participants will all be managed by the contract research organization (CRO) 83 Bar, Inc.

Study interventions will include CBD formulation or a placebo control that will be taken orally (p.o.) as two small capsules one hour prior to going to bed. CBD formulation is a capsule made to GMP standards composed of vegetable cellulose (gluten-free, non-GMO, vegan, certified kosher, certified halal) that contains 300 mg >99.9% purity hemp-derived CBD made in a GMP-certified laboratory that is dissolved in coconut oil (USDA certified organic, non-GMO, dairy-free, gluten-free, vegan coconut oil). CBD formulation is a custom chemical formulation that also contains low concentrations (1 mg each) of highly purified (>98% purity) forms of the terpenes linalool, myrcene, phytol, limonene, α-terpinene, α-terpineol, α-pinene, and β-caryophyllene [Food Grade (GRAS), Natural, Organic, non-GMO, dairy-free, gluten-free, vegan) made in GMP-certified laboratories. CBD formulation contains no Δ-9-tetrahydrocannabinol (Δ-9-THC). The safety of CBD1 and these terpenes at the doses found in CBD formulation is well established.

### Study Duration

The estimated time from when Le Rêve 3.0 opens enrollment until completion of data analyses will be 20 weeks.

Participant Duration: After enrollment, the participant duration for the entire study will be 12 weeks.

SCHEMA of the clinical study is shown in FIG. 12.

TABLE 2
Procedures and timeline

| | Screening | | Collect | | Treatment | | Treatment | Post- |
|---|---|---|---|---|---|---|---|---|
| | Weeks −4 | Enrollment | Baseline Data | Treatment 1 | Washout | Treatment 2 | Washout | Treatment |
| Procedures | to −1 | Day 1 | Weeks 1-2 | Weeks 3-6 | Week 7 | Weeks 8-11 | Week 12 | Weeks 13-20 |
| Informed consent | X | | | | | | | |
| Medical history | X | | | | | | | |
| Randomization | | | | X | | | | |
| Administer study | | | | X | | X | | |
| intervention | | | | | | | | |
| Adverse event | | | | | | | | X |
| review and | | | | | | | | |
| evaluation | | | | | | | | |

TABLE 3
Objective and Endpoints

| | | JUSTIFICATION |
|---|---|---|
| OBJECTIVES | ENDPOINTS | FOR ENDPOINTS |
| Primary | | |
| The primary objective is to | The primary endpoint is the | Deep and REM sleep |
| determine if CBD formulation | percentage of time spent in slow | are thought to |
| influences objective measures of | wave (Deep) and REM sleep as | contribute significantly |
| sleep physiology. | quantified by a non-invasive wrist- | to restorative sleep. |
| | worn sleep-tracking device called | An unbiased approach is |
| | Whoop. | used to collect objective |
| | | data on these sleep |
| | | stages to determine if |
| | | Defined CBD increases |
| | | deep and REM sleep. |
| Secondary | | |
| The secondary objective(s) are to | Additional objective secondary | Although some early |
| determine if CBD formulation | outcome measures that will be | clinical studies suggest |
| influences objective and subjective | collected on the Whoop device | that CBD might help |
| measures of sleep physiology. | include: Total sleep time, sleep | people with insomnia, |
| | latency, the number of sleep | no studies have |
| | disturbances, time spent in each | addressed if it |
| | sleep stage, and the percentage of | influences objective |
| | time spent in each sleep stage. | and subjective |
| | A modified version of a clinically | measures of sleep |
| | validated questionnaire entitled the | physiology. Therefore, |
| | Participant Global Impression (PGI)¹⁴ | a number of objective |
| | will be used to track subjective | and subjective |
| | measures of sleep physiology. This | secondary outcome |
| | data will be collected from study | measures in this study |
| | participants in the form of a brief | to fill this void in |
| | five-minute survey that will be | knowledge. |
| | completed on their smartphone at | |
| | the end of each four-week treatment | |
| | period. This survey measures the | |
| | perception of the study participants | |
| | of the effects of treatment. | |

### Study Population

In order to be eligible to participate in this study, an individual must meet all of the following criteria: has provided a signed and dated informed consent form; presence of chronic insomnia defined as self-reported difficulty initiating (latency to persistent sleep >30 min) and/or maintaining sleep (>30 mins awake during the middle of the night, or waking >30 mins before desired waking time on three or more nights per week) for at least 3 months; insomnia Severity Index score >15.

Male or female aged 25-70 years.

Is willing to comply with all study procedures throughout the entire study, including:
Wearing a sleep-tracking device on their wrist throughout entire clinical study.
Ensuring that the sleep-tracking device is connected to their smartphone via Bluetooth on a daily basis so that sleep data can be collected on a daily basis.
Ensuring that the sleep-tracking device is charged before going to bed each night, ensuring that sleep data can be collected on a daily basis.
Is willing to receive and respond to daily text (SMS) notifications for the duration of the entire clinical study.
During the treatment phases of the study, is willing to take the treatment for at least four nights in each week.
On the nights in which the participant takes the treatment, is willing to abstain from excessive alcohol intake (>two drinks/day).

Female subjects who: are postmenopausal, with amenorrhea for at least one year before the screening interview, OR are surgically sterile, OR; if of childbearing potential agree to practice effective methods of contraception, from the time of the signing of informed consent through the last dose of study treatment, or agree to completely abstain from intercourse; self-reported bedtime between 9 pm and midnight on four-seven nights per week; and owns a smartphone.

### Lifestyle Considerations

During this study, participants are asked to: refrain from ingesting greater than two standard drinks/day of alcohol consumption for study duration; abstain from ingesting greater than 400 mg/day of caffeine consumption for study duration; abstain from ingesting caffeine after 3 pm for study duration; abstain from ingesting more than five cups or glasses per day of xanthine-containing beverages (i.e., tea, coffee, energy drinks or cola) for study duration.

### 6.1 Study Intervention(s) Administration

CBD formulation is composed of small capsules made with vegetable cellulose (gluten-free, non-GMO, vegan, certified kosher, certified halal) that contain >99.9% purity hemp-derived CBD dissolved in coconut oil (USDA certified organic, non-GMO, dairy-free, gluten-free, vegan coconut oil) with a custom chemical formulation that also contains highly purified (>98% purity) forms of the terpenes linalool, myrcene, phytol, limonene, α-terpinene, α-terpineol, α-pinene, and β-caryophyllene [Food Grade (GRAS), Natural, Organic, non-GMO, dairy-free, gluten-free, vegan). Participants will be required to take two small capsules one hour before they go to bed. These capsules will contain a total of 300 mg CBD and 8 mg of terpenes (1 mg of each terpene). CBD formulation contains no Δ-9-THC. The capsules that contain the placebo control will look, feel and smell exactly the same as the capsules that contain CBD formulation, but will contain no CBD or terpenes.

The dose participants will take in the study is two small capsules, which contain a total of 300 mg CBD and 8 mg of terpenes (1 mg each of the terpenes linalool, myrcene, phytol, limonene, α-terpinene, α-terpineol, α-pinene, and β-caryophyllene) that will be taken orally with a glass of water one hour before bedtime. The participants will be required to take the study medication on a minimum of four nights per week for each of the four-week treatment phases of the study.

### Dosing

The dose participants will take in the study is two small capsules, that contain a total of 300 mg CBD and 8 mg of terpenes (1 mg each of the terpenes linalool, myrcene, phytol, limonene, α-terpinene, α-terpineol, α-pinene, and β-caryophyllene) that will be taken orally with a glass of water one hour before bedtime. The participants will be required to take the study medication on a minimum of four nights per week for each of the four-week treatment phases of the study.

### Efficacy Assessments

150 study participants will be enrolled who display chronic insomnia defined as a self-reported difficulty initiating (latency to persistent sleep >30 min) and/or maintaining sleep (>30 min awake, or waking >30 min before desired waking time) on three or more nights per week for at least 3 months. Participants will also complete a brief clinically validated insomnia severity index, and only participants with an Insomnia Severity Index score >15 will be enrolled in Le Rêve 3.0. Males or females were enrolled aged 25-70 years that are located in the United States that meet inclusion and exclusion criteria.

Le Rêve 3.0 will be conducted in an entirely decentralized manner. Primary data for Le Rêve 3.0 will be obtained in a completely unbiased manner from a non-invasive sleep-tracking wrist-worn device called “Whoop” (https://www.whoop.com), that electronically collects and transmits sleep data from study participants in the comfort of their own beds.

The wristband collects hundreds of data points per second from a 3-axis accelerometer, 3-axis gyroscope, and heart rate sensor. The wristband can accurately measure the latency to fall asleep, total sleep time, sleep fragmentation, as well as the time spent in each sleep stage [Light, Slow Wave (Deep), Rapid Eye Movement (REM), and Awake]. The device also collects data using photoplethysmography (PPG), a technique that involves measuring blood flow by assessing superficial changes in blood volume. Heart rate, heart rate variability and respiratory rate, can all be derived from PPG data, and all of these metrics are used in Whoop's sleep detection and staging algorithms. Importantly, two recent publications, published independently from Whoop, show that data on sleep stages collected from the Whoop device are highly accurate and correlate well with polysomnography (PSG), the gold-standard of sleep tracking used in clinical studies conducted in sleep clinics.

The primary endpoint efficacy endpoint in Le Rêve 3.0 is the percentage of time spent in slow wave (Deep) and REM sleep as collected by a non-invasive wrist-worn sleep-tracking device called Whoop.

Additional objective secondary outcome measures that will be collected on the Whoop device include: Total sleep time, sleep latency, the number of sleep disturbances, time spent in each sleep stage, and the percentage of time spent in each sleep stage.

A modified version of a clinically validated questionnaire entitled the PGI19 will be used to track subjective measures of sleep physiology. This data will be collected from study participants in the form of a brief five-minute survey that will be completed on their smartphone at the end of each four-week treatment period. This survey measures the perception of the study participants of the effects of treatment.

### Example 2: A Double-Blind, Placebo-Controlled, Randomized, Crossover Investigational Study to Determine if Defined CBD, a Custom-Formulated Cannabidiol (CBD) Capsule, Influences Sleep Physiology

### Summary

126 participants were enrolled, 63 completed study. Initial analysis of study data indicates primary endpoint of clinical trial [percentage of time participants spent in SWS+REM (restorative) sleep] showed a statistically significant positive result. The secondary endpoint of percentage of time participants spent in SWS sleep was also met. Data is independently analyzed to confirm the initial findings.

### Study Description

Insomnia is a disorder in which people have inadequate or poor-quality sleep due to a number of factors, such as difficulty falling asleep, waking up frequently during the night with difficulty returning to sleep, waking up too early in the morning, or having unrefreshing sleep. Defined CBD (labeled as CBD-Terpenes in the figures) is a capsule composed of highly purified (>99.9% purity) hemp-derived CBD and terpenes (>98% purity). Defined CBD contains no Δ-9-tetrahydrocannabinol (Δ-9-THC). The research study, entitled Le R\vec{e}ve 3.0, is designed to evaluate the efficacy of Defined CBD on sleep physiology. This trial is not intended to treat, diagnose, prevent, or cure any disease.

Primary Objectives are to determine if Defined CBD influences objective measures of sleep physiology. Secondary Objectives are to determine if Defined CBD influences subjective measures of sleep physiology.

Primary Endpoint is the percentage of sleep spent in deep [slow wave sleep (SWS)] and rapid eye movement (REM) sleep as determined using an objective wrist-worn sleep-tracking device called Whoop. Secondary Endpoints include the total sleep time, sleep latency, the number of sleep disturbances, time spent in each sleep stage, percentage of time spent in each sleep stage, and subjective measures of sleep physiology as collected via participant surveys.

150 study participants will be enrolled who display chronic insomnia defined as a self-reported difficulty initiating (latency to persistent sleep >30 min) and/or maintaining sleep (>30 min awake, or waking >30 min before desired waking time) on three or more nights per week for at least 3 months. Participants will also complete a brief clinically validated insomnia severity index, and only participants with an Insomnia Severity Index score >15 will be enrolled in Le Rêve 3.0. Males or females aged 25-70 years will be enrolled that are located in the United States that meet inclusion and exclusion criteria.

Le Rêve 3.0 will be a decentralized study completed by participants in the comfort of their own homes. Recruitment, enrollment, informed consent, and distribution of study materials to participants will all be managed by the contract research organization (CRO) 83 Bar, Inc.

### Description of Study Interventions

Study interventions will include Defined CBD or a placebo control that will be taken orally (p.o.) as two small capsules one hour prior to going to bed. Defined CBD is a capsule made to GMP standards composed of vegetable cellulose (gluten-free, non-GMO, vegan, certified kosher, certified halal) that contains 300 mg >99.9% purity hemp-derived CBD made in a GMP-certified laboratory that is dissolved in coconut oil (USDA certified organic, non-GMO, dairy-free, gluten-free, vegan coconut oil). Defined CBD is a custom chemical formulation that also contains low concentrations (1 mg each) of highly purified (>98% purity) forms of the terpenes linalool, myrcene, phytol, limonene, α-terpinene, α-terpineol, α-pinene, and α-caryophyllene [Food Grade (GRAS), Natural, Organic, non-GMO, dairy-free, gluten-free, vegan) made in GMP-certified laboratories. Defined CBD contains no Δ-9-tetrahydrocannabinol (Δ-9-THC). The safety of CBD and these terpenes at the doses found in Defined CBD is well established.

Study Duration is the estimated time from when Le Rêve 3.0 opens enrollment until completion of data analyses will be 20 weeks. Participant Duration for the entire study will be 12 weeks after enrollment.

### Results

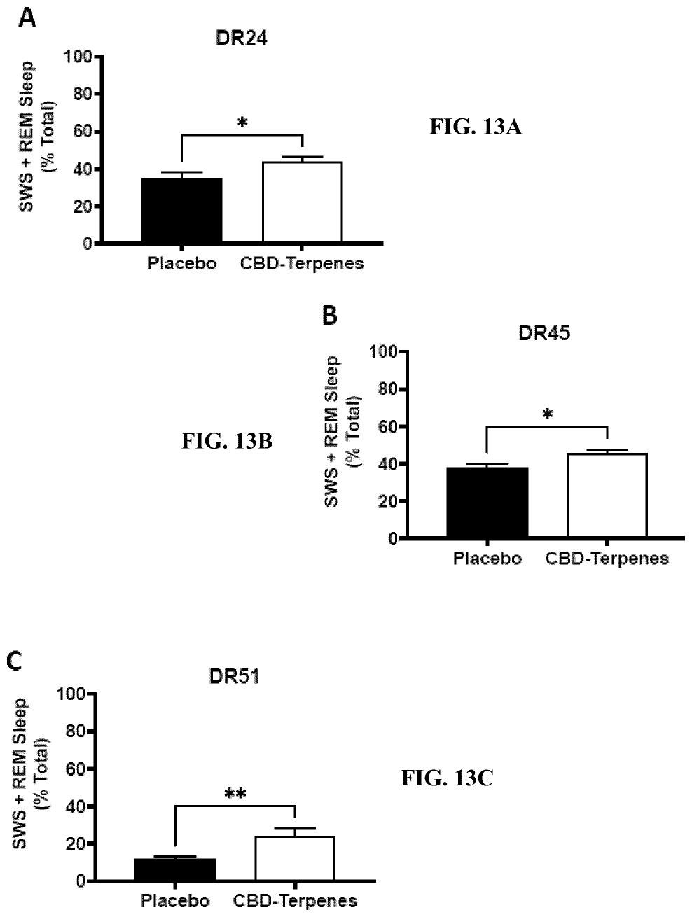

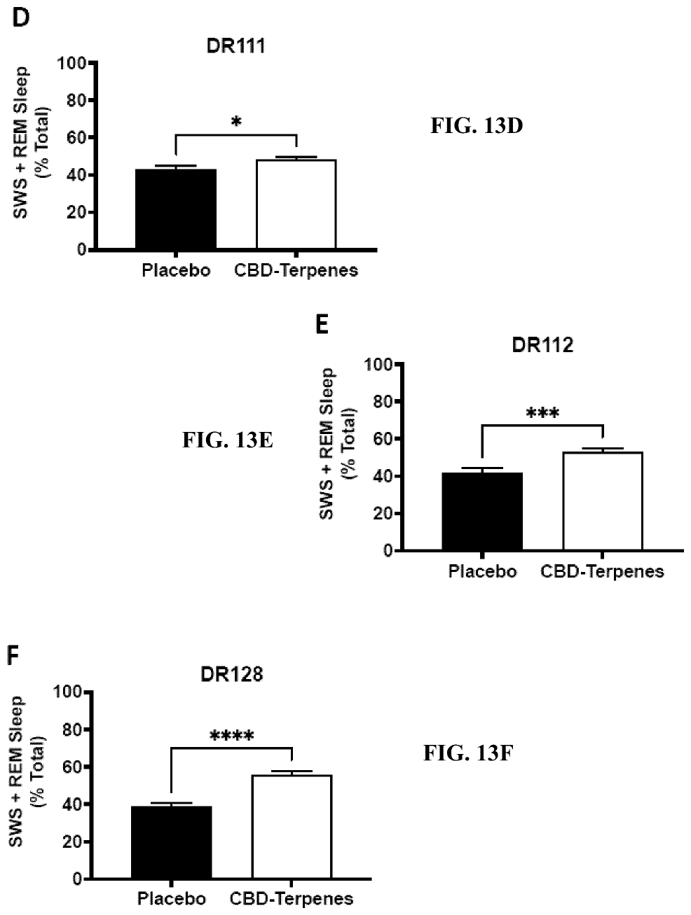

FIGS. 13A-13F are graphs showing the CBD-terpenes formulation increases the percentage of time DR24 (FIG. 13A), DR45 (FIG. 13B), DR51 (FIG. 13C), DR111 (FIG. 13D), DR112 (FIG. 13E), and DR128 (FIG. 13F) spends in SWS+REM sleep (restorative sleep) [*p<0.05 (t test); **p<0.01 (t test); ***p<0.001; ****p<0.0001 (t test). n=15-28 days per treatment group. Values are means±SEM]. Participant DR24 showed an increase in SWS+REM sleep of about 3% compared to placebo; participant DR45 showed an increase in SWS+REM sleep of about 3%; participant DR51 showed an increase in SWS+REM sleep of about 7%; participant DR111 showed an increase in SWS+REM sleep of about 3%; participant DR112 showed an increase in SWS+REM sleep of about 9%; and participant DR128 showed an increase in SWS+REM sleep of about 13%.

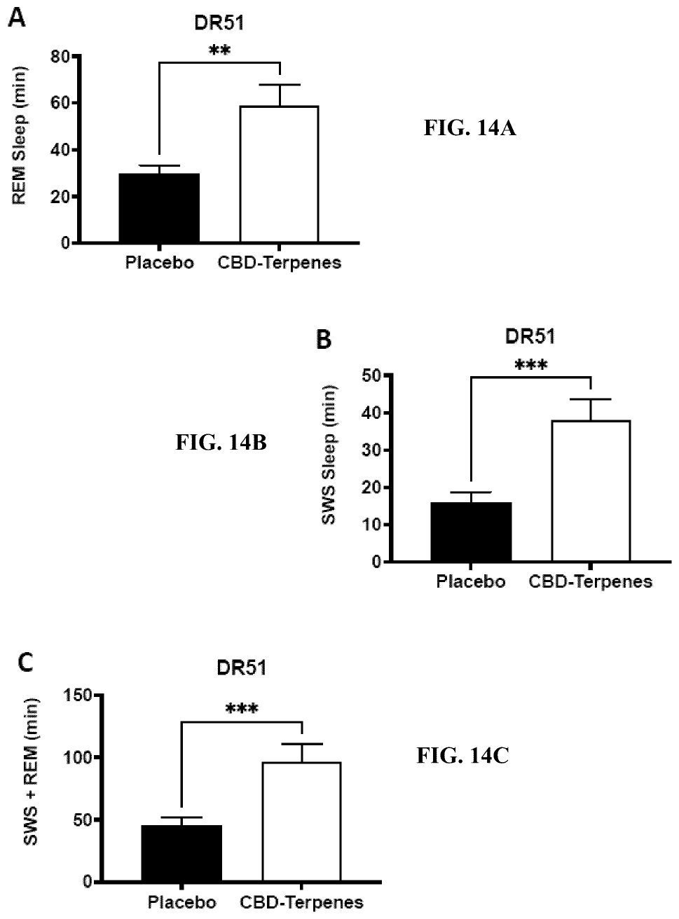

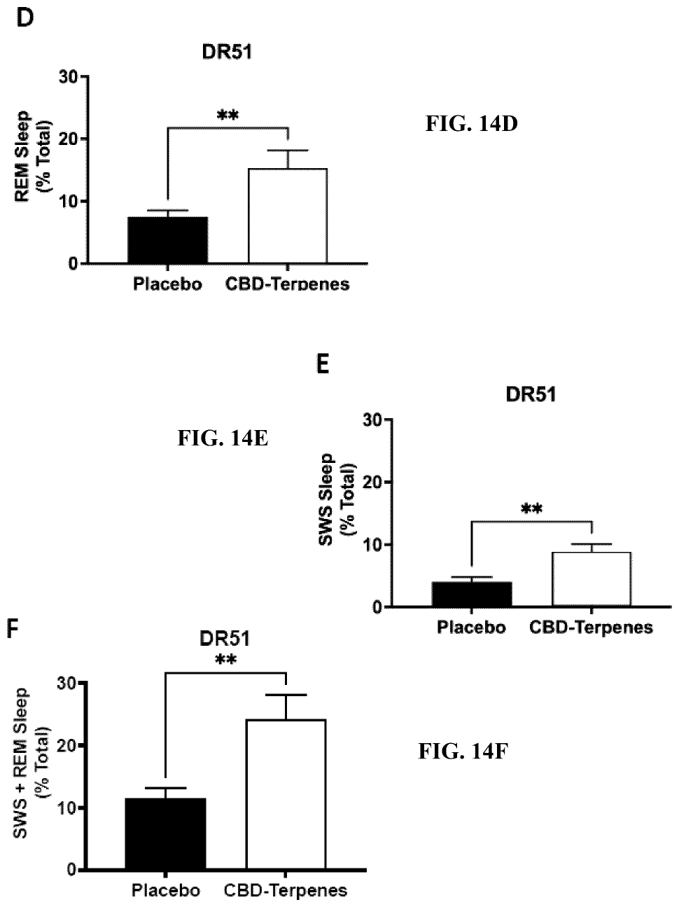

FIGS. 14A-14F are graphs showing the CBD-terpenes formulation increases (FIG. 14A) REM, (FIG. 14B) SWS, (FIG. 14C) SWS+REM (restorative sleep) in study participant DR51, as well as the percentage of time DR51 spends in (FIG. 14D) REM and (FIG. 14E) SWS, and (FIG. 14F) SWS+REM (restorative sleep) [**p<0.01; ***p<0.001 (t test). n=19-25 days per treatment group. Values are means±SEM]. Participant DR51 showed an increase in REM sleep of about 30 minutes compared to placebo; participant DR51 showed an increase in SWS sleep of about 22 minutes; participant DR51 showed an increase in SWS+REM sleep of about 51 minutes; participant DR51 showed an increase in REM sleep of about 7%; participant DR51 showed an increase in SWS sleep of about 5%; and participant DR51 showed an increase in SWS+REM sleep of about 13%.

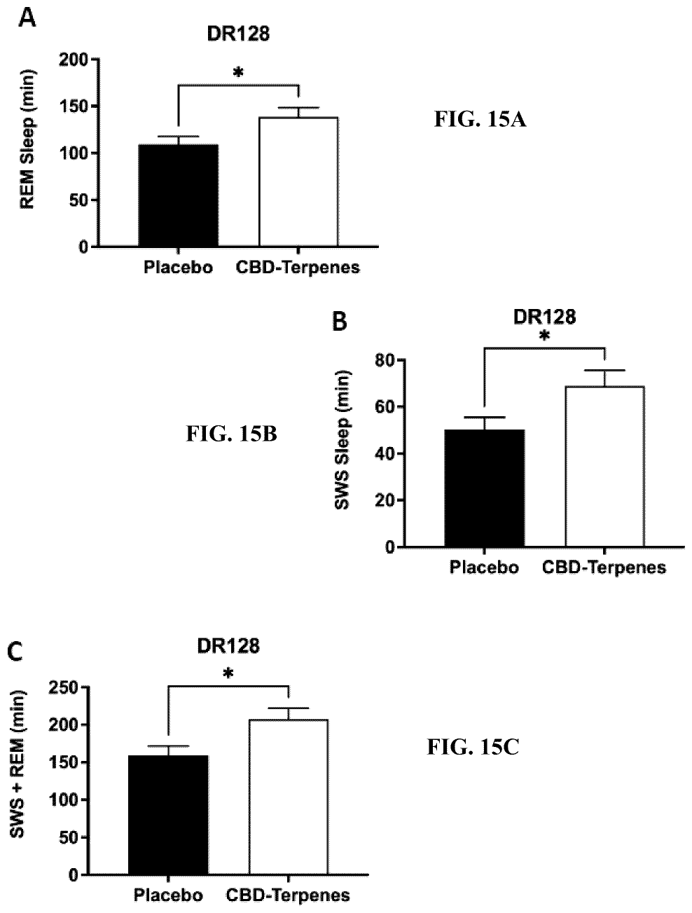

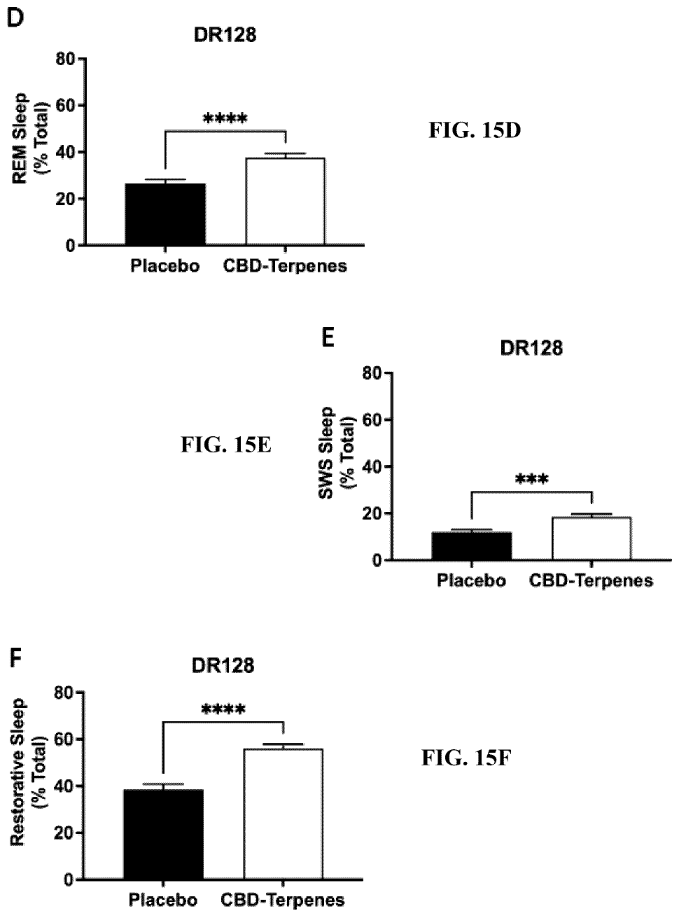

FIGS. 15A-15F are graphs showing the CBD-terpenes formulation increases (FIG. 15A) REM, (FIG. 15B) SWS, (FIG. 15C) SWS+REM (restorative sleep) in study participant DR128, as well as the percentage of time DR128 spends in (FIG. 15D) REM and (FIG. 15E) SWS, and (FIG. 15F) SWS+REM (restorative sleep) *p<0.05 (t test); ***p<0.001; ****p<0.0001 (t test). n=15-28 days per treatment group. Values are means±SEM]. Participant DR128 showed an increase in REM sleep of about 30 minutes compared to placebo; participant DR128 showed an increase in SWS sleep of about 19 minutes; participant DR128 showed an increase in SWS+REM sleep of about 50 minutes; participant DR128 showed an increase in REM sleep of about 13%; participant DR128 showed an increase in SWS sleep of about 8%; and participant DR128 showed an increase in SWS+REM sleep of about 18%.

Six participants who self-identified as having insomnia and also scored as having severe clinical insomnia (>22) on a clinically validated insomnia severity index¹³ were administered capsules containing 300 mg of highly purified CBD and 8 mg of terpenes, or a placebo control, a minimum of four nights per week for a period of four weeks. After a one week washout period, participants were administered the second treatment (i.e., capsules with CBD-terpenes or the placebo control), for a second four week treatment period.

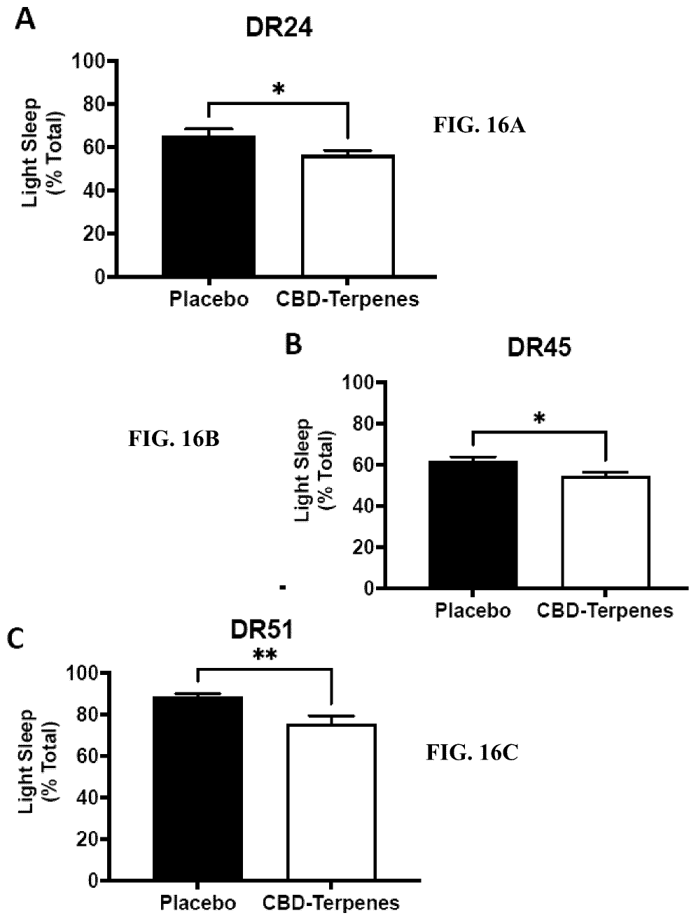

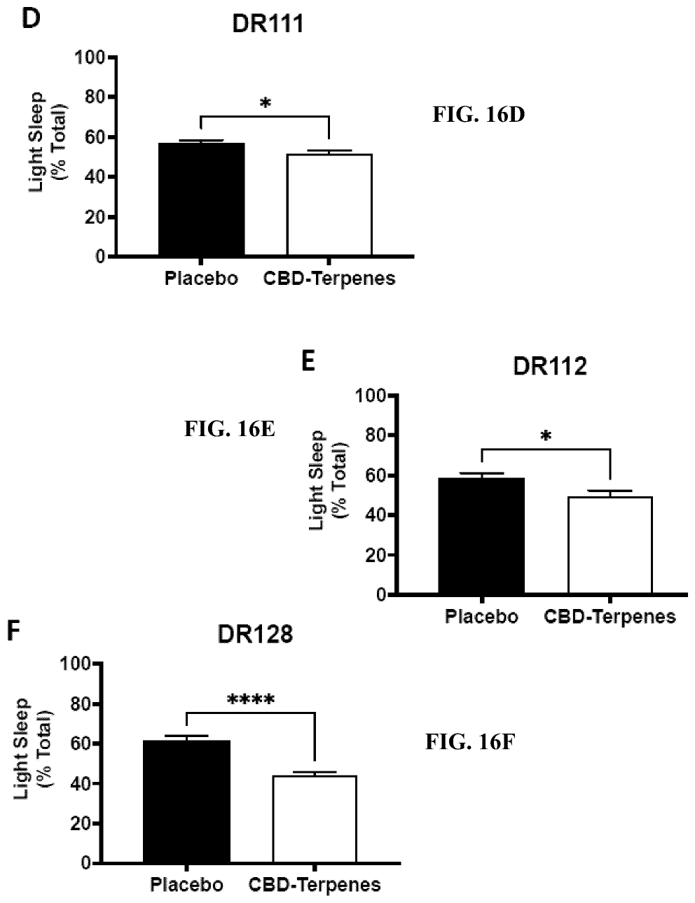

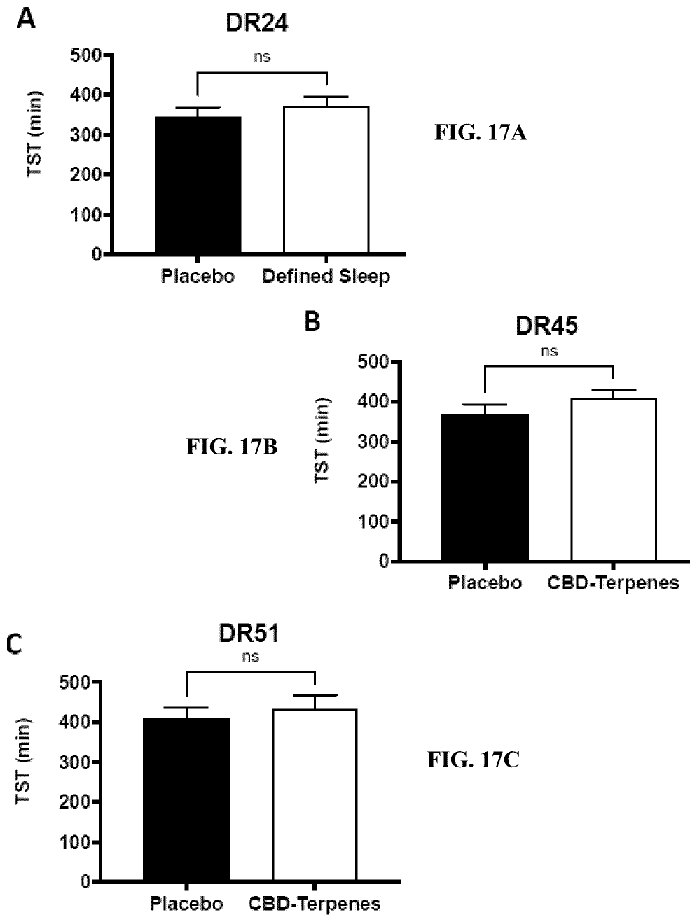

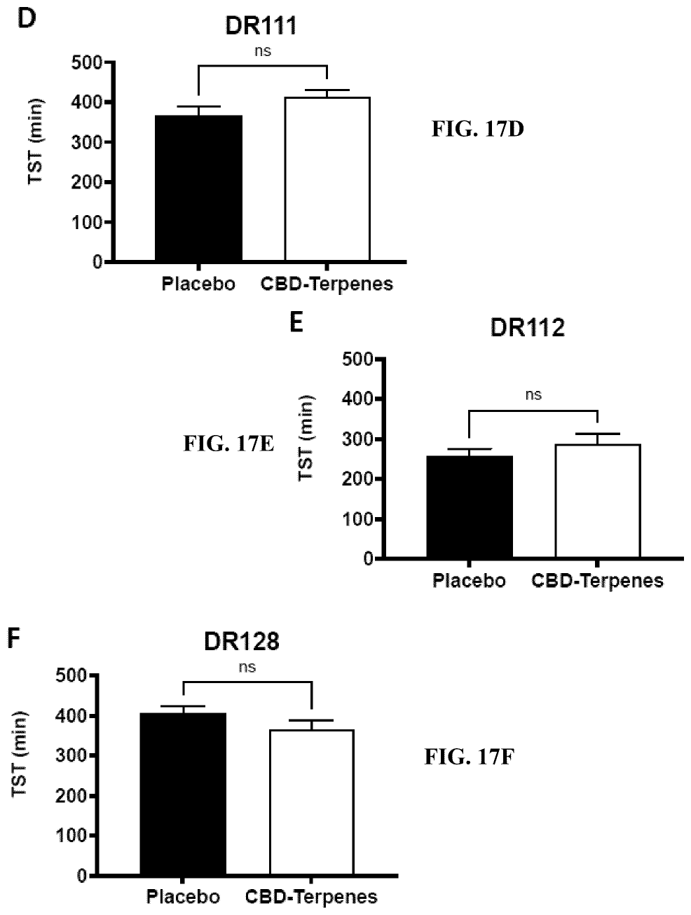

Capsules that contained CBD-terpenes significantly increased the percentage of time all six participants spent in SWS+REM sleep (restorative sleep) as quantified with a clinically validated^{14,15} wrist-worn sleep tracking device (FIG. 13A-13F). The magnitude of the increased percentage of restorative sleep in these participants varied from 5-18%, representing an absolute increase of 38-51 min per night over a period of one month (an absolute increase of ˜19-26 h of restorative sleep over the one month treatment period). Moreover, capsules with CBD-terpenes significantly decreased the percentage of time spent in light sleep in all of the six participants relative to the placebo control (FIGS. 16A-16F), but had no effect on total sleep time (FIGS. 17A-17F).

In participants DR51 and DR128, the CBD-terpene capsules also significantly increased the absolute time spent in REM, SWS, and SWS+REM sleep, as well as the percentage of time spent in REM, SWS and SWS+REM sleep (FIGS. 14 and 15). In participant DR51, who only averaged 45 min of SWS+REM sleep per night on the placebo control, treatment with the CBD-terpene capsules increased SWS+REM sleep by an average of 52 min per night (an average increase of >6 h per week of restorative sleep or >24 h per four week treatment period) (FIG. 15). Similarly, in participant DR128 the CBD-terpene capsules increased SWS+REM sleep by 49 min per night (an average increase of ˜6 h per week or >24 h per four week treatment period) (FIG. 14). No adverse events were reported by any participants during this study.

### DISCUSSION

The use of CBD in humans and animals has increased dramatically in the past decade. Although consumers use CBD to ostensibly self-treat a variety of common ailments, including chronic pain, anxiety and insomnia, sparse clinical evidence supports its use to effectively treat any of these ailments. Moreover, while medical practitioners are increasingly asked by their patients if they should use CBD to treat these ailments, in the absence of rigorous clinical research, doctors are unable to provide any informed guidance on the proper use of CBD to their patients.

In this case series, we report that daily administration of capsules which contain highly purified CBD and terpenes significantly increase the percentage of time six participants with severe clinical insomnia spend in SWS+REM sleep. The combination of SWS and REM sleep stages is commonly referred to as “restorative” sleep¹⁶. Numerous studies indicate that SWS and REM sleep stages are critically important for strengthening the immune system, increasing cell and tissue regeneration, increasing brain metabolite clearance, replenishing energy stores, and for learning, memory, attention and executive function^{16,17}. SWS is associated with decreased heart rate, blood pressure, sympathetic nervous activity and cerebral glucose utilization, compared with wakefulness¹⁶. Moreover, during SWS, human growth hormone is released while the stress hormone cortisol is inhibited¹⁶. Notably, the percentage of time spent in SWS and REM sleep decreases with aging^{17,18}. While clinical studies have shown that many commonly prescribed sleep medications including barbiturates, benzodiazepines, zolpidem, zopiclone and neuroactive steroids reduce sleep latency, many of these medications decrease SWS and REM sleep¹⁹.

The CBD-containing capsules used in this case series also contain small quantities (1 mg each) of terpenes. These small molecules, found commonly in plants, are currently classified by the FDA as Generally Regarded As Safe (GRAS) for human consumption and are commonly used as flavoring additives in food. While preclinical studies in animals show that high concentrations of the terpenes used in this study are sedating in animals^{8-12,20}, their potential effects on sleep physiology in humans have not been established, and will be the subject of future investigations.

The capsules with CBD-terpenes used in this study contained no detectable Δ-9-THC. Although a recent study suggests that treatment with a complex mixture of THC, CBD and other cannabinoids might improve some subjective sleep outcomes in patients with insomnia²¹, causal relationships are difficult to infer from this study, given that THC induces euphoria and increases heart rate at the concentrations that were tested²². It is difficult to claim that this study was truly placebo-controlled when the participants could discern which treatment arm had the active treatment. Notably, this study showed no effect of cannabinoid treatment on objective measures of sleep physiology²².

In summary, the results from this case series indicate that capsules which contain a highly purified CBD-terpene mixture that lacks Δ-9-THC significantly increases the percentage of time six patients with insomnia spent in restorative sleep stages. We did not observe any adverse indications in the course of this case series, and current research to date indicates that CBD has a relatively good safety profile²³, especially relative to the majority of commonly prescribed sleep medications, which have clearly documented and serious side effect profiles²⁴. If the results reported in this case series can be replicated in rigorous placebo-controlled clinical trials, the current results suggest CBD might represent a safer alternative as a potential treatment for insomnia that boosts, rather than decreases, restorative sleep.

### PUBLICATIONS

1—Ford et al., Sleep Med. 2015; 16(3):372-8.
2—Chagas et al., J Psychopharmacol. 2013; 27(3):312-6.
3—Carlini and Cunha, J Clin Pharmacol. 1981; 21(S1):4175-4275.
4—Shannon et al, Perm J 2019; 23:18-041.
5—Linck et al., Phytomedicine. 2009; 16(4):303-7.
6—Buchbauer et al., J Pharm Sci. 1993; 82(6):660-4.
7—Lee et al., Biomol Ther (Seoul). 2018; 26(4): 368-373.
8—do Vale et al., Phytomedicine. 2002; 9(8):709-14.
9—Freitas et al., Braz J Med Biol Res. 1993; 26(5):519-23.
10—Tan et al., Trends Neurosci. 2011; 34(4):188-197.
11—Yang et al., Mol Pharmacol. 2016; 90(5):530-539.
12—Costa et al., Brain Res. 2014; 1547:34-42.
13—Kessler et al., Mol Nutr Food Res. 2014; 58(4):851-862.
14—Rodrigues de Almeida Costa et al., Journal of Ethnopharmacology. 2011; 137(1):828-836.
15—Watt et al., Eur J Pharmacol. 2008; 590(1-3):120-126.
16—Bang et al., Archives of Pharmacal Res. 2002; 25:643-646.
17—Zhou et al., J Nutr Sci Vitaminol (Tokyo). 2009; 55(4):367-373.
18—Elisabetsky et al., Fitoterapia. 1995; 66(5):407-414.
19—Galdino et al., Prog Neuropsychopharmacol Biol Psychiatry. 2012; 38(2):276-284.
20—Bahi et al., Physiol Behav. 2014; 135:119-124.
21—Sharpe et al., J Transl Med. 2020; 18:374-395.
22—Bergamaschi et al., Neuropsychopharmacol. 2011; 36(6):1219-1226.
23—Zuardi et al., Psychopharmacol. 1982; 76(3):245-250.
24—Fusar-Poli et al., Arch Gen Psychiatry. 2009; 66(1):95-105.
25—Berryhill et al., J Clin Sleep Med. 2020; 16(5):775-783.
26—Miller et al., Journal of Sports Sciences. 2020; 38(22):2631-2636.
27—Buysse et al., Psychiatry Res. 1989; 28(2):192-213.
28—Krystal et al., Sleep. 2008; 31:79-90.
29—Hamilton, Br J Med Psychol. 1959; 32:50-55.
30—Krause et al., Nat Rev Neurosci. 2017; 18(7):404-418.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of treating a sleep disorder, comprising the steps:
a. providing a formulation comprising a CBD and a plurality of terpenes including linalool, myrcene, phytol, limonene, α-terpinene, α-terpineol, α-pinene, and β-caryophyllene; and
b. administering an effective dose of the formulation to the subject to act on the subject's sleep disorder.

2. The method of claim 1, wherein the CBD comprises about 150 mg of CBD and about 0.5 mg each of the plurality of terpenes and no Δ-9-tetrahydrocannabinol.

3. The method of claim 2, further comprising increasing REM sleep or restorative sleep in the subject.

4. The method of claim 3, wherein the percentage of increase in REM sleep is at least 20%.

5. The method of claim 4, wherein the administering step is once daily, one hour before bedtime, for at least seven consecutive nights.

6. The method of claim 4, wherein the administering step is orally as a capsule at least one hour prior to going to bed.

7. The method of claim 6, wherein the administering step is for a 4 week period and the increase in slow wave sleep (SWS)+rapid eye movement (REM) sleep is increased by an average of 50 minutes per night over the 4 week period.

8. The method of claim 7, wherein the administering step significantly decreases the percentage of time spent in light sleep for the subjects.

9. A method of treating anxiety, comprising the steps:
a. providing a formulation comprising a CBD and a plurality of terpenes including linalool, myrcene, phytol, limonene, α-terpinene, α-terpineol, α-pinene, and β-caryophyllene; and
b. administering an effective dose of the formulation to the subject to act on the subject's anxiety.

10. The method of claim 9, wherein the CBD comprises about 150 mg of CBD and about 0.5 mg each of the plurality of terpenes.

11. The method of claim 10, wherein the administering step is once daily, one hour before bedtime, for at least seven consecutive nights.

12. The method of claim 11, wherein the administering step lowers the subject's anxiety score according to the Hamilton Anxiety Rating Scale.

13. A method of treating depression, comprising the steps:
a. providing a formulation comprising a CBD and a plurality of terpenes including linalool, myrcene, phytol, limonene, α-terpinene, α-terpineol, α-pinene, and β-caryophyllene; and
b. administering an effective dose of the formulation to the subject to act on the subject's depression.

14. The method of claim 13, wherein the CBD comprises about 150 mg of CBD and about 0.5 mg each of the plurality of terpenes.

15. The method of claim 14, wherein the administering step is once daily, one hour before bedtime, for at least seven consecutive nights.
